(19) United States
(12) Patent Application Publication
(10) Pub. No.: US 2026/0053389 A1
(43) Pub. Date: Feb. 26, 2026

(54) INGESTIBLE CAPSULE WITH ON-BOARD SENSOR HARDWARE

(71) Applicant: Atmo Biosciences Limited, Box Hill (AU)

(72) Inventors: James JOHN, Box Hill (AU); Malcolm HEBBLEWHITE, Box Hill (AU); Adam CHRIMES, Box Hill (AU); Kyle BEREAN, Box Hill (AU); William KENTLER, Box Hill (AU)

(21) Appl. No.: 19/105,642

(22) PCT Filed: Aug. 22, 2023
(86) PCT No.: PCT/AU2023/050801

§ 371 Date: Feb. 21, 2025

(30) Foreign Application Priority Data
Aug. 23, 2022 (AU) 2022902414

(51) Int. Cl. A61B 5/07; A61B 5/00; A61B 5/01

(52) U.S. Cl. CPC A61B 5/073; A61B 5/002; A61B 5/01; A61B 5/4255; A61B 5/6861; A61B 2562/0219; A61B 2562/029

(57) ABSTRACT

Embodiments include an ingestible capsule being configured, in response to identification of an excretion marker from on-board sensor hardware, to determine occurrence of an excretion event, and in response to determining occurrence of the excretion event to modify one or more settings of the wireless data transmitter to start, restart, increase transmission power of, or increase a rate of, wireless transmission of the data transmission payload away from the ingestible capsule by a wireless data transmitter.

### TECHNICAL FIELD

This invention relates to sensors useful in an ingestible sensor capsule for medical and health applications in the gastrointestinal (GI) tract of mammals including humans, and specifically relates to processing signals from a sensor housed in an ingestible capsule and wirelessly transmitting processing results.

### BACKGROUND

Sensor capsules such as that disclosed in EP3497437A1 house gas sensors and other sensors within an ingestible capsule so that readings may be made from within the gastrointestinal (GI) tract of a mammal, from which readings information about the GI tract may be determined, such as motility reports and concentrations of analyte gases.

A process for determining type and concentration of particular gases in a multi-gas mixture based on readings taken from within the GI tract by gas sensors on-board an ingestible capsule is disclosed in EP3619526A1.

There are many reports on the strong likelihood of the association of these gas constituents to different illnesses, conditions, etc, and to the association of gases with gut health and therefore overall wellbeing.

However, in order to associate the gas constituents to different illnesses, conditions, etc, it is desirable to know or to be able to accurately predict a location of the capsule within the GI tract at the instant a gas sensor reading is obtained. Certain gases or concentrations of gases that would be expected in one part of the GI tract may be an indicator of an illness or condition in another location within the GI tract. Furthermore it is desirable to monitor and report on health indicators such as GI tract motility. Motility in the present context refers to progress of an ingestible object such as an ingestible capsule through the gastrointestinal tract, such as may be represented by residence time in different GI tract regions.

Furthermore, information relating to the motility of the capsule (i.e. representing the progression of the capsule through the GI tract, for example, by determining time taken to passage various GI tract organs) may be used in diagnosis of conditions and assessment of GI tract and gut health.

Gut health is increasingly identified as a contributor to overall health and wellness. Motility of an ingestible capsule (with or without associated gas constituent measurements) provides important information in the assessment of gut health.

Accurate prediction and determination of capsule location provides information in itself as a record of motility of the capsule which provides useful indicators of GI tract health and condition. Furthermore, a contemporaneous record of signals from one or more other sensors, or data extracted therefrom, can be combined with the motility record to provide further indicators of GI tract health and condition.

It is desirable to address limitations in the state of the art regarding reliable transmission, storage, and/or processing of signals provided by sensors on-board an ingestible sensor capsule.

### STATEMENTS

Embodiments include an ingestible capsule comprising: an ingestible indigestible bio-compatible housing; and further comprising, within the housing: a power source; sensor hardware including a temperature sensor configured to output a temperature sensor signal representing a temperature of an environment surrounding the ingestible capsule; processor hardware; memory hardware; and a wireless data transmitter; wherein, during a passage of the ingestible capsule through a GI tract of the subject mammal, the processor hardware is configured to receive a signal being output by the sensor hardware, to process the received signal, and to store some or all of the processed signal, or data extracted therefrom, on the memory hardware as data transmission payload; the processor hardware is configured to receive and monitor the temperature sensor signal to identify when the temperature sensor signal indicates that the ingestible capsule ceases to be within the GI tract of the subject mammal, and in response to said identification to determine occurrence of an excretion event, and in response to determining occurrence of the excretion event to modify one or more settings of the wireless data transmitter to start, restart, increase transmission power of, or increase a rate of, wireless transmission of the data transmission payload away from the ingestible capsule by the wireless data transmitter.

Optionally, the data transmission payload transmitted away from the capsule in response to determining occurrence of the excretion event, comprises a report that occurrence of the excretion event has been determined.

Optionally, modifying the settings causes the wireless data transceiver to transmit in a broadcast or inquiry mode the report that occurrence of the excretion event has been determined to a receiver device, and to wirelessly connect, or to wirelessly re-connect following an initial connection pre-ingestion of the ingestible capsule, to the receiver device to transmit the remainder of the data transmission payload.

Optionally, the sensor hardware includes one or more gas sensors, and therein the signal output by the sensor hardware and processed by the processor hardware includes a gas sensor signal output by the one or more gas sensors, and the data transmission payload includes the processed gas sensor signal or data extracted therefrom.

Optionally, the one or more gas sensors includes one or more from among:
one or more spectrophotometers;
one or more Surface Acoustic Wave sensors;
one or more Bulk Acoustic Resonator Arrays;
one or more VOC gas sensors; and
one or more TCD gas sensors;
each of the one or more gas sensors configured to generate a component gas sensor signal forming part of the gas sensor signal.

Optionally, processing the received gas sensor signal comprises identifying one or more motility event indicators in the received gas sensor signal, and storing a representation of the identified motility indicators on the memory hardware as data transmission payload.

Optionally, identifying the one or more motility event indicators comprises monitoring the gas sensor signal received from each of the one or more gas sensors in a most recent time period of predefined duration on a rolling basis to identify a spike, step change, or inflection in the gas sensor signal as the motility indicator.

Optionally, the one or more gas sensors comprises one or more from among a VOC gas sensor and a TCD gas sensor, and the gas sensor signal comprises one or more from among a VOC gas sensor signal and a TCD gas sensor signal, accordingly.

Optionally, the sensor hardware further comprises one or more from among: an accelerometer; a reflectometer formed by an antenna in series with a directional coupler, wherein the antenna is an antenna of the data transmitter, the antenna being controlled by the processor to transmit an intermittent or continuous signal from which a reflectometer signal is obtainable; wherein further to identifying the motility event indicator in the gas sensor signal, the processor hardware is configured to: store, in association with the motility event indicator in the data transmission payload, a representation of a signal received contemporaneously with the motility event indicator from one or more sensors within the housing from among: a gas sensor other than the gas sensor providing the signal in which the motility event indicator is detected; the accelerometer; or the reflectometer.

Optionally, the representation of the signal is one or more from among:
a recording of the signal,
a recording of the signal downsampled by retaining only one in every more than one readings;
a dimensionally reduced version of the signal;
a recording of a confirmatory marker identified by processing the signal;
a characteristic value of the signal obtained by processing the signal, the characteristic value being an average value, a rate of change, a maximum, a local maximum, a minimum, or a local minimum.

Optionally, the ingestible capsule further comprises an accelerometer, and during passage through the GI tract the processor hardware is configured to receive an accelerometer signal output by the accelerometer, to process the received accelerometer signal, and to store the processed accelerometer signal or a representation thereof on the memory hardware as data transmission payload.

Optionally, to determine occurrence of the excretion event in response to identifying the temperature represented by the temperature sensor signal dropping below the predefined range of temperatures for the subject mammal, the processor hardware is configured to determine whether or not the received accelerometer signal indicates the ingestible capsule experiencing a freefall event, and if it is determined that the received accelerometer signal indicates the ingestible capsule experiencing a freefall event, the processor hardware is configured to determine that the excretion event has occurred.

Optionally, the processor hardware is configured to process the temperature sensor signal, and to store the processed temperature signal or data extracted therefrom on the memory hardware as data transmission payload.

Optionally, the wireless data transmitter is a Bluetooth transceiver.

Optionally, the wireless data transmitter is a Bluetooth transceiver configured to operate according to a Bluetooth Low Energy Coded PHY transmission protocol.

Optionally, the Bluetooth transceiver comprises an integrated radio and a microcontroller.

Optionally, modifying one or more settings of the wireless data transmitter in response to determining occurrence of the excretion event includes controlling the Bluetooth transceiver to transmit the data transmission payload stored on the memory hardware by broadcasting data pending transmission from the data transmission payload to a recipient device irrespective of whether or not the recipient device is paired to the Bluetooth transceiver.

Optionally, preceding determining the occurrence of the excretion event, the Bluetooth transceiver is configured to pair with a Bluetooth compatible device external to the subject mammal and to transfer to the paired device data comprising or representing one or more from among: a signal or signals from the sensor hardware; one or more motility indicators identified by processing signals from the sensor hardware; one or more identified diagnostic indicators identified by processing signals from the sensor hardware; information representing remaining capacity of the power source; a metric calculated by processing a signal from a single sensor among the sensor hardware or by combining signals from plural sensors among the sensor hardware; calculated gas concentration levels for one or more constituent gases among a gas mixture present in the GI tract, calculated by reference to predefined calibration parameters stored on the memory hardware; and proceeding determination of occurrence of the excretion event the processor hardware is configured to modify settings of the Bluetooth transceiver to transfer the data transmission payload to the same Bluetooth compatible device, either by continuing the existing pairing, by re-pairing, or in the absence of pairing.

Optionally, the ingestible capsule is configured, following an initiation event, to operate in an ingestion detection mode in which the temperature sensor is operational, and the processor hardware is operational and monitoring the temperature sensor signal to identify when the temperature represented by the temperature sensor signal reaches a predefined range of internal temperatures for the subject mammal, and in response to said identification to determine occurrence of an ingestion event, to record the ingestion event on the memory hardware as data transmission payload, and to begin collecting data during the passage through the GI tract.

Embodiments may include an ingestible capsule comprising:
an ingestible indigestible bio-compatible housing;
and further comprising, within the housing:
   a power source;
   sensor hardware;
   processor hardware;
   memory hardware;
   a Bluetooth transceiver;
wherein, during a passage of the ingestible capsule through a GI tract of the subject mammal, the processor hardware is configured to receive a signal being output by the sensor hardware, to process the received signal by calculating a metric representing the received signal or by identifying a motility indicator or a diagnostic indicator in the received signal, and to store the calculated metric or data representing the motility indicator or the diagnostic indicator on the memory hardware as data transmission payload, the motility indicator and/or the diagnostic indicator being a predefined pattern, value range, spike, step change, inflection point, local maximum or local minimum, predefined change or sequence of changes, in a signal or signals output by one or more sensors among the sensor hardware; the Bluetooth transceiver being configured to transmit the data transmission payload away from the ingestible capsule to a paired recipient device during the passage through the GI tract of the subject mammal.

Embodiments may include an ingestible capsule comprising: an ingestible indigestible bio-compatible housing; and further comprising, within the housing: a power source; sensor hardware; processor hardware; memory hardware; and a wireless data transmitter; the ingestible capsule being configured, following ingestion by a subject mammal, to collect data during a passage through a GI tract of the subject mammal, during which passage the processor hardware is configured to receive a signal output by the sensor hardware, to process the received signal, and to store some or all of the processed signal, or data extracted therefrom, on the memory hardware as data transmission payload; the processor hardware is configured to receive and monitor the signal output by the sensor hardware to identify a transmission trigger event indicator, and in response to said identification to determine occurrence of a transmission trigger event, and in response to determining occurrence of the transmission trigger event to modify one or more settings of the wireless data transmitter to start, restart, increase transmission power of, or increase a rate of, wireless transmission of the data transmission payload away from the capsule by the wireless data transmitter.

Embodiments may include a method in an ingestible capsule adapted for ingestion by a subject mammal, the ingestible capsule comprising: an ingestible indigestible bio-compatible housing; and, within the housing: a power source; sensor hardware including a temperature sensor configured to output a temperature sensor signal representing a temperature of an environment surrounding the ingestible capsule; processor hardware; memory hardware; and a wireless data transmitter; the method comprising: at the ingestible capsule, following ingestion of the ingestible capsule by the subject mammal, collecting data during a passage through a GI tract of the subject mammal; during the passage through the GI tract of the subject mammal, receiving a signal output by the sensor hardware, processing the received signal, and storing some or all of the processed signal, or data extracted therefrom, on the memory hardware as data transmission payload; at the processor hardware, receiving and monitoring the temperature sensor signal to identify when the temperature sensor signal indicates that the ingestible capsule ceases to be within the GI tract of the subject mammal, and in response to said identification, determining occurrence of an excretion event, and in response to determining occurrence of the excretion event, modifying one or more settings of the wireless data transmitter to start, restart, increase transmission power of, or increase a rate of, wireless transmission of the data transmission payload away from the ingestible capsule by the wireless data transmitter.

Embodiments may include a method in an ingestible capsule, the ingestible capsule comprising:
an ingestible indigestible bio-compatible housing;
and further comprising, within the housing:
   a power source;
   sensor hardware;
   processor hardware;
   memory hardware;
   a Bluetooth transceiver;
   the method comprising, following ingestion of the ingestible capsule by a subject mammal:
      at the processor hardware, receiving a signal being output by the sensor hardware, processing the received signal by calculating a metric representing the received signal or by identifying a motility indicator or a diagnostic indicator in the received signal, and storing the calculated metric or data representing the motility indicator or the diagnostic indicator on the memory hardware as data transmission payload, the motility indicator and/or the diagnostic indicator being a predefined pattern, value range, spike, step change, inflection point, local maximum or local minimum, predefined change or sequence of changes, in a signal or signals output by one or more sensors among the sensor hardware; at the Bluetooth transceiver, transmitting the data transmission payload away from the ingestible capsule to a paired recipient device during the passage through the GI tract of the subject mammal.

Embodiments may include a method in an ingestible capsule, the ingestible capsule comprising:
an ingestible indigestible bio-compatible housing;
and further comprising, within the housing:
   a power source;
   sensor hardware;
   processor hardware;
   memory hardware; and
   a wireless data transmitter;
the method comprising, following ingestion of the ingestible capsule by a subject mammal, during a passage through a GI tract of the subject mammal, at the processor hardware, receiving a signal being output by the sensor hardware, processing the received signal, and storing some or all of the processed signal, or data extracted therefrom, on the memory hardware as data transmission payload; at the processor hardware, receiving and monitoring the signal output by the sensor hardware to identify a transmission trigger event indicator, and in response to said identification, determining occurrence of a transmission trigger event, and in response to determining occurrence of the transmission trigger event, modifying one or more settings of the wireless data transmitter to start, restart, increase transmission power of, or increase a rate of, wireless transmission of the data transmission payload away from the capsule by the wireless data transmitter.

Embodiments may include a computer program for execution by processor hardware in an ingestible capsule adapted for ingestion by a subject mammal, the ingestible capsule comprising: an ingestible indigestible bio-compatible housing; and, within the housing: a power source; sensor hardware including a temperature sensor configured to output a temperature sensor signal representing a temperature of an environment surrounding the ingestible capsule; the processor hardware; memory hardware; and a wireless data transmitter; the computer program, when executed by the processor hardware, causes the processor hardware to perform a method comprising: following ingestion of the ingestible capsule by the subject mammal, during a passage through the GI tract of the subject mammal, receiving a signal being output by the sensor hardware, processing the received signal, and storing some or all of the processed signal, or data extracted therefrom, on the memory hardware as data transmission payload; receiving and monitoring the temperature sensor signal to identify when the temperature sensor signal indicates that the ingestible capsule ceases to be within the GI tract of the subject mammal, and in response to said identification, determining occurrence of an excretion event, and in response to determining occurrence of the excretion event, modifying one or more settings of the wireless data transmitter to start, restart, increase transmission power of, or increase a rate of, wireless transmission of the data transmission payload away from the ingestible capsule by the wireless data transmitter.

Embodiments may include a computer program for execution by processor hardware in an ingestible capsule, the ingestible capsule comprising: an ingestible indigestible bio-compatible housing; and further comprising, within the housing: a power source; sensor hardware; the processor hardware; memory hardware; a Bluetooth transceiver; the computer program, when executed by the processor hardware, causes the processor hardware to perform a method comprising: following ingestion of the ingestible capsule by a subject mammal, during a passage through a GI tract of the subject mammal, at the processor hardware, receiving a signal output by the sensor hardware, processing the received signal by calculating a metric representing the received signal or by identifying a motility indicator or a diagnostic indicator in the received signal, and storing the calculated metric or data representing the motility indicator or the diagnostic indicator on the memory hardware as data transmission payload, the motility indicator and/or the diagnostic indicator being a predefined pattern, value range, spike, step change, inflection point, local maximum or local minimum, predefined change or sequence of changes, in a signal or signals output by one or more sensors among the sensor hardware; at the Bluetooth transceiver, transmitting the data transmission payload away from the ingestible capsule to a paired recipient device during the passage through the GI tract of the subject mammal.

Embodiments may include a computer program for execution by processor hardware in an ingestible capsule, the ingestible capsule comprising:
an ingestible indigestible bio-compatible housing;
and further comprising, within the housing:
   a power source;
   sensor hardware;
   the processor hardware;
   memory hardware; and
   a wireless data transmitter;
the computer program, when executed by the processor hardware, causes the processor hardware to perform a method comprising, during a passage of the ingestible capsule through a GI tract of a subject mammal, receiving a signal being output by the sensor hardware, processing the received signal, and storing some or all of the processed signal, or data extracted therefrom, on the memory hardware as data transmission payload; receiving and monitoring the signal output by the sensor hardware to identify a transmission trigger event indicator, and in response to said identification, determining occurrence of a transmission trigger event, and in response to determining occurrence of the transmission trigger event, modifying one or more settings of the wireless data transmitter to start, restart, increase transmission power of, or increase a rate of, wireless transmission of the data transmission payload away from the capsule by the wireless data transmitter.

### BRIEF DESCRIPTION OF DRAWINGS

Embodiments are discussed below, by way of example, with reference to the accompanying drawings, in which:

FIG. 1A is a schematic of an ingestible capsule;

FIG. 1B is a schematic of the electronic components of an ingestible capsule;

FIG. 1C is a system including an ingestible capsule;

FIG. 2 illustrates a schematic of the electronic components of an ingestible capsule;

FIG. 3 illustrates changing sensitivity to constituent gases with operating temperature;

FIG. 4 is a flow of processing according to an embodiment;

FIG. 5 is a flow of processing according to an embodiment;

FIG. 6 illustrates data and processing operations in generation of a motility report according to an embodiment;

FIG. 7a illustrates a plot of data generated by an embodiment;

FIG. 7b illustrates a plot of data generated by an embodiment;

FIG. 7c illustrates a plot of data generated by an embodiment;

FIG. 8a illustrates a plot of data generated by an embodiment;

FIG. 8b illustrates a plot of data generated by an embodiment;

FIG. 8c illustrates a plot of data generated by an embodiment;

FIG. 8d illustrates a plot of data generated by an embodiment;

FIG. 9a illustrates a plot of data generated by an embodiment;

FIG. 9b illustrates a plot of data generated by an embodiment;

FIG. 9c illustrates a plot of data generated by an embodiment;

FIG. 9d illustrates a plot of data generated by an embodiment;

FIG. 10 illustrates a flow of processing according to an embodiment;

FIG. 11 illustrates a flow of processing according to an embodiment; and

FIG. 12 illustrates a flow of processing according to an embodiment.

### DETAILED DESCRIPTION

### Ingestible Capsule Overview

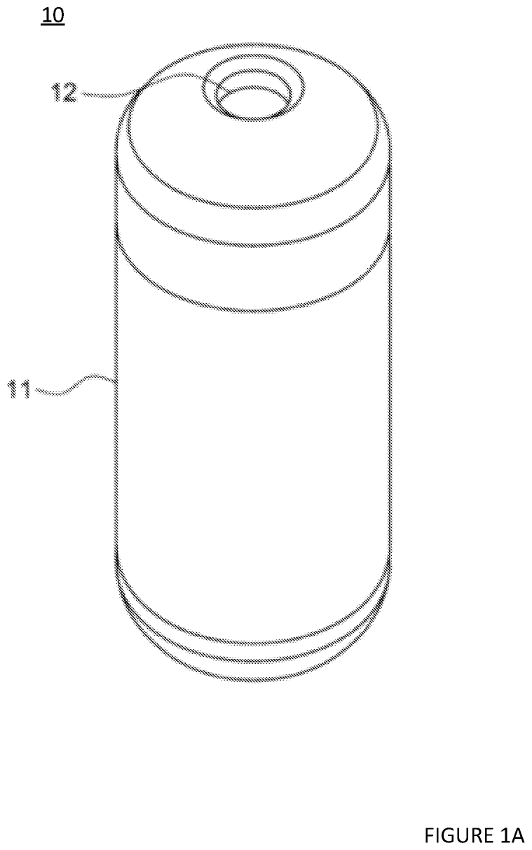

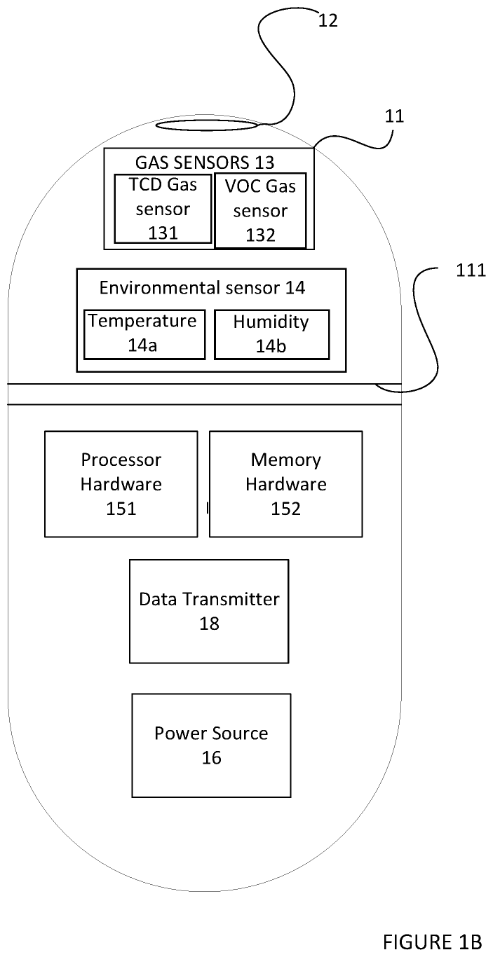

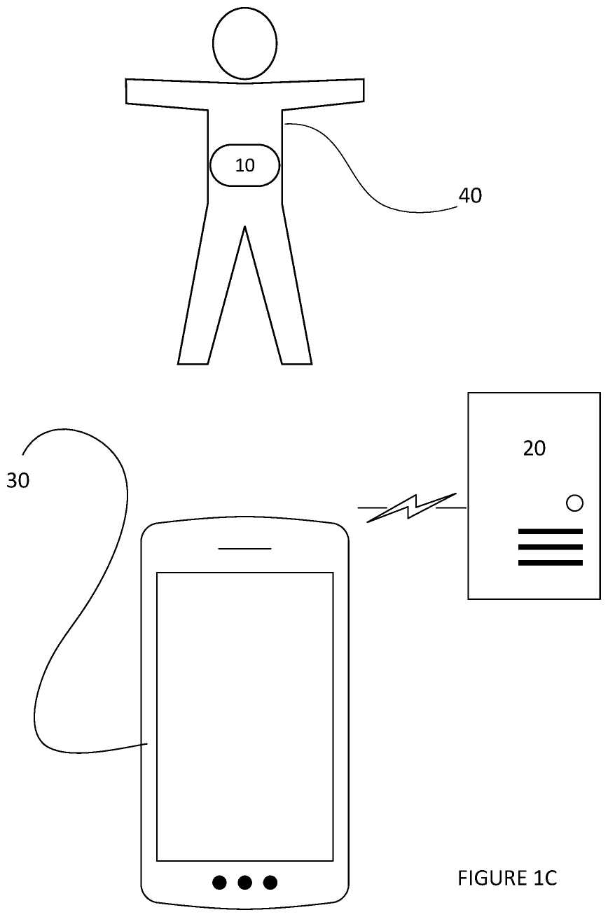

FIGS. 1A and 1B illustrate an ingestible capsule 10. A system including the ingestible capsule 10 of FIGS. 1A and 1B is illustrated in FIG. 1C, during a live phase of the ingestible capsule 10 (i.e. while the ingestible capsule 10 is obtaining readings from within the GI tract of a subject mammal 40).

As shown in FIGS. 1A and 1B the typical capsule 10 consists of a housing such as a gas impermeable shell 11 which has an opening covered by a gas permeable membrane 12. A membrane 111 separates an exposed interior cavity exposed to the environmental gases entering the capsule 10 through the membrane 12 from a sealed-off interior cavity that is not exposed to the environmental gases.

As shown in FIG. 1C, the system, in addition to the capsule, further comprises a receiver apparatus 30 which receives data transmitted by the capsule from within the GI tract of the subject mammal during the live phase. Concurrently or subsequently, the receiver apparatus 30 processes the received data and may also upload some or all of the received data to a remote processing apparatus such as a cloud-based service for further processing. The remote computer may be a cloud resource, or may be a standalone computer at a clinician premise at which the subject is a patient, or may be a server (be it cloud-based or otherwise) at a service provider to which the clinician is a subscriber/customer/servicer user.

Optionally, a system may further comprise a remote processing apparatus 20 such as a server forming part of a cloud computing environment or some other distributed processing environment. The remote processing apparatus 20 may be a server provided by or on behalf of a clinical centre at which subject 40 is a patient and taking responsibility for interpreting the results generated by the capsule 10 (i.e. the data transmission payload) and reporting them to the subject 40.

Connectivity between the capsule 10 and the receiver apparatus 30 is via the data transmitter on the capsule, which may be part of a wireless transceiver, for example a Bluetooth transceiver, which may operate according to a standard Bluetooth transmission protocol or according to Bluetooth Low Energy transmission protocol. Other operable communication technologies include LoRa, wifi and 433 MHz radio.

Internally the capsule 10 includes gas sensor hardware 131, 132, an environmental sensor 14, and processor hardware 151 and memory hardware 152. The processor hardware 151 and memory hardware 152 may be a microcontroller. The processor hardware 151 may be a microprocessor. The memory hardware 152 may be a non-volatile memory and the data stored thereon is accessible by the processor hardware 151. The processor hardware 151 processes data from signals received from the gas sensor hardware and the environmental sensor (and optionally also the reflectometer and accelerometer) and stores the processed data on the memory hardware 152. The processed data, or a portion thereof, is stored on the memory hardware 152 as a data transmission payload ready for transmission to a receiver apparatus 30 by the data transmitter 18.

By way of example, the capsule illustrated in FIG. 1C houses, as sensor hardware, an environmental sensor 14 in the form of a temperature sensor 14a and/or a humidity sensor 14b, gas sensors in the form of a TCD gas sensor 131 and a VOC gas sensor 132, an accelerometer 19, and a reflectometer. Embodiments may include any single or combination of those individual sensors. Alternatively or additionally, embodiments may include one or more sensors not illustrated in FIG. 1C such as a spectrophotometer, Surface Acoustic Wave sensor, and/or Bulk Acoustic Resonator Arrays.

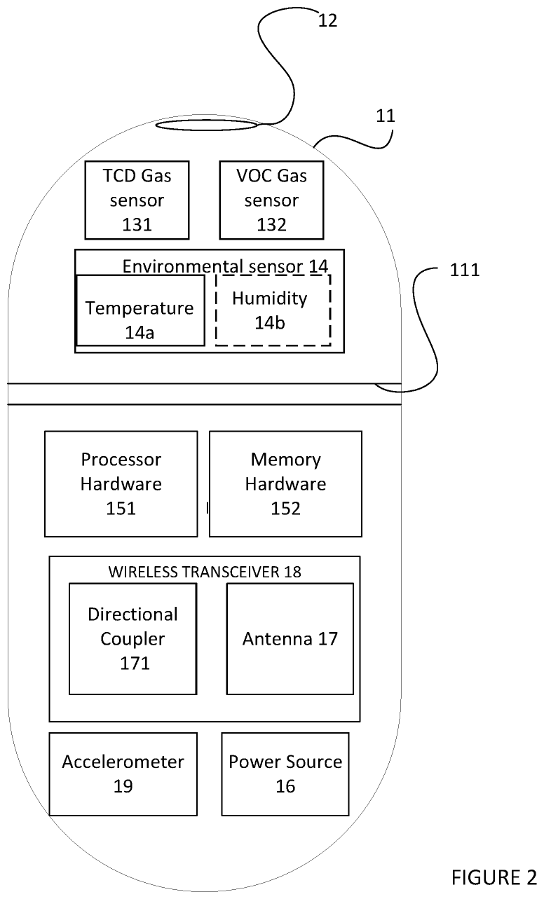

The environmental sensor 14 may be a temperature sensor 14a or may be a temperature sensor 14a and a humidity sensor 14b. The gas sensors may be a TCD gas sensor 131, a VOC gas sensor 132, or a TCD gas sensor 131 and a VOC gas sensor 132. As illustrated in FIG. 2, the internal electronics may also include a power source 16, for example, silver oxide batteries, an antenna 17, a wireless transceiver 18. The internal electronics may also include a reed switch. Other options for keeping the device switched off (or otherwise not consuming power) during storage include a physical switch pressed via a flexible part of the housing, or a photodetector and coupled field effect transistor that latches the microcontroller on when exposed to light. The internal electronics may further comprise an accelerometer 19 from which accelerometer data (i.e. a signal) is received at the processor hardware 151 for processing and subsequent storage at the memory hardware 152 and transmission by the data transmitter 18.

The gas sensors 131, 132 are less than several mm in dimension each and are sensitive to particular gas constituents including oxygen, hydrogen, carbon dioxide and methane. In fact, the VOC gas sensor 132 may be configured to give sensor side readings and driver or heater side readings. The heater side readings may be used to determine thermal conductivity of a surrounding gas and thereby the heater side readings of the VOC gas sensor are TCD readings. The sensor side readings are used to determine concentrations of volatile organic compounds in the surrounding gases and are VOC readings. The TCD gas sensor 131 may be, for example, a heating element coupled to a thermopile output, with the thermopile temperature, and therefore its output, varying due to energy conducted into the gas at the location of the capsule 10. The TCD gas sensor 131 measures rate of heat diffusion away from the heating element.

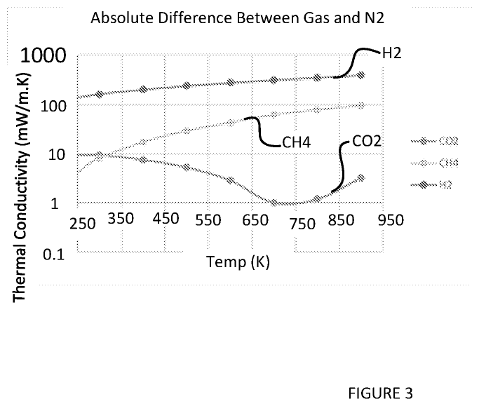

As illustrated in FIG. 3, the heater side of the VOC gas sensor 132 (operating as a TCD sensor) and the sensor side of the TCD gas sensor 131 have different operating ranges, so TCD readings from the two sensors collectively span a wider range of operating temperatures than either of the sensors individually. Both sensors have heating elements. The TCD gas sensor 131 has a low operating temperature but with a high precision. The heater side of the VOC gas sensor 132 increases the operating range but has a lower precision for TCD readings than the TCD sensor. The larger collective thermal range achieved by the two gas sensors 13 in concert enables better resolution of analytes in the event that the signals from the gas sensors are processed to resolve the analytes. The thermal conductivity of constituent gases in the gas mixture of the GI tract varies with temperature and so by obtaining TCD readings at different operating temperatures the different gases can be resolved from each other. This is leveraged in a gas resolution processing branch, which is to determine identity and concentrations of constituent gases in the gas mixture surrounding the capsule 10. The gas resolution processing may be performed on-board the gas capsule 10, at the receiver apparatus 30, or at a remote processing apparatus. The gas resolution processing is optional depending on the implementation.

The gas sensors 13 are contained in a portion of the capsule 10 sealed from the power source 16 and other electronic components by a membrane 111. Such an arrangement minimises volume of the sensing headspace (i.e. the sealed portion) and minimises risk of a leak caused by a perforated membrane allowing GI-tract gases from the headspace to reach the power source. However, since the power source may be configured so that exposure to GI-tract gases does not adversely impact performance, the membrane may be omitted. That is, the membrane 111 is optional. The membrane 111 is permeable by electronic circuitry required to connect the components housed on either side. For example, wiring may pass through the membrane 111 in a sealed manner. The outer surface of the sealed portion of the capsule is composed of a selectively permeable membrane. Selectively permeable in the present context indicates that liquids are not allowed to permeate whereas gases are. The selectivity does not extend to allowing only a subset of gases to permeate. For example, the gas sensors 13 include respective heaters which are driven to heat sensing portions of the respective gas sensors 13 to temperatures at which sensor readings are obtained (i.e. a measurement temperature). The heaters may be driven in pulses so that there is temporal variation in the sensing portion temperature and so that measurement temperatures are obtained for periods sufficient to take readings but without consuming the power that would be required to sustain the measurement temperature continuously.

The gas sensors 13 are calibrated, so that a gas sensor reading can be used to identify the composition and concentration of a gas to which they are exposed. Calibration coefficients are gathered in manufacturing and testing and are applied to the recorded readings at the processing stage (i.e. by a server such as on the cloud). Otherwise, this calibration could be performed on the capsule 10, at the receiver apparatus 30, or on any device having access to the calibration coefficients and the recorded readings from the gas sensors 13. Such calibration relates to a gas resolution branch of processing concerned with measuring the concentration of constituent gases in the gas mixture at the capsule 10. Context for the outputs of that branch of processing is provided by a motility branch of processing, which determines (or predicts to within predefined confidence level) a location of the capsule 10 within the GI tract at which said gas mixture is found. In the motility (or location determination) processing branch, some calibration may also be required in seeking to find gastric-duodenal transition indicators, since ingested foodstuffs at different temperatures change the environmental temperature in the stomach, which influences rate of heat diffusion. In the case of gas sensor readings taken after ingestion and before the gastric-duodenal transition (i.e. whilst the capsule 10 is in the stomach), processing of readings may include applying a moderation to TCD readings, from either gas sensor, in order to correct for variations in environmental temperature, based on environmental temperature readings by the temperature sensor 14a. TCD readings are effectively measuring rate of heat loss to surroundings, and so accuracy is improved by measuring the temperature of the surroundings rather than by relying on assumption (i.e. prior knowledge of internal temperature of the subject mammal). However, the processing may rely on assumption, for example, if there is some issue with the temperature sensor readings, or, for example, if the level of accuracy provided by assumption is acceptable in a particular implementation. Gastric temperature may vary based on, for example, ingestion of liquids or foodstuffs by the subject mammal, or physical activity undertaken by the subject mammal 40. Environmental temperature is a term used in this document to refer to the temperature of the environment in which the capsule 10 is located, as distinct from operational temperatures of the gas sensors 13. The sensitivity of the gas sensors 13 to different constituent gases vary according to the operating temperature of the sensors and the processing of the readings includes calibrating (also referred to as moderating or correcting) readings from the gas sensors according to contemporaneous operating temperature and optionally also according to contemporaneous environmental temperature.

It is noted that the motility branch of processing and the gas resolution branch of processing are not independent of one another. Some motility indicators (i.e. features or characteristics of sensor output signals used to determine timing of motility events) may be found in readings of concentration of a single analyte gas in the gas mixture at the capsule, obtained by processing the output of one or more of the gas sensors 13.

In addition to the gas sensors 13 and the temperature sensor 14a, the capsule electronics further include processor hardware 151, memory hardware 152, a power source 16, an antenna 17, a wireless transmitter 18, and optionally a reed switch. The wireless transmitter 18 operates in concert with the antenna 17 to transmit readings from the sensors (collectively referring to the gas sensors 13 and the temperature sensor 14a, and optionally also the accelerometer 19 and reflectometer) to a receiver apparatus 30 for processing thereon or at a remote processing apparatus to which the receiver apparatus is in data communication, or the processor hardware 151 processes the signals received from the sensors to identify motility indicators (or otherwise to extract information from the sensor readings).

The wireless transmitter 18 (also referred to as data transmitter 18) may be provided as part of a wireless transceiver 18. The wireless transceiver 18 includes an antenna 17. Optionally, the wireless transceiver 18 also includes a directional coupler 171. The wireless transceiver 18 may transmit data in accordance with the Bluetooth protocol, the Bluetooth Long Range (coded-PHY) protocol, the LoRa protocol, the wifi protocol, or using another mode of transmission such as 433 MHz radio wave transmission.

In the example of a Bluetooth wireless transceiver 18, in the pre-excretion transmission technique the transceiver may be operated according to a long-range or coded-PHY Bluetooth transmission procedure, such as BTLE Coded PHY. A signal power enhancement of around 10 dB is achievable via BTLE Coded PHY Bluetooth transmission procedure.

FIG. 2 illustrates the antenna 17 and directional coupler 171 as elements of the wireless transmitter 18, since the antenna is the physical means by which the wireless transmitter 18 transmits data to the receiver apparatus 30. The wireless transmitter 18 is also configured to buffer data for transmission. The wireless transmitter 18 may also be configured to encode the data with a code unique to the capsule 10 among a population of like capsules 10.

Interconnections between electronic components in FIG. 3 may be via a central bus. This is one example of how power and data may be distributed between components. Other circuitry architecture may be implemented, for example, all connections may be via a microcontroller which coordinates distribution of data and power between components. The sensors (the TCD sensor 131, the VOC sensor 132, the temperature sensor 14a, the accelerometer 19, and the directional coupler 171) take readings under the instruction of a microcontroller, powered by the power source 16, and transfer the readings (or results of processing the readings) to the wireless transmitter 18 for transmission to the receiver apparatus via the antenna 17. For example, the processor hardware 151 and memory hardware 152 may collectively be referred to as a microcontroller.

The dimension of the capsule may be less than 11.2 mm in diameter and less than 27.8 mm in length. The housing of the capsule 10 may be made of indigestible polymer, which is biocompatible. The housing may be smooth and non-sticky to allow its passage in the shortest possible time and to minimise risk of any capsule retention. Optionally, the ingestible capsule may be less than 32.3 mm in length and less than 11.6 mm in diameter.

The antenna 17 may be in series with a directional coupler 171. The directional coupler 171 and the antenna 17 are configured as a reflectometer. The reflectometer measures the amplitude of reflected signals by means of a diode detector. The measurements of the reflectometer are readings that represent electromagnetic properties of material in the vicinity of the capsule. The reflectometer readings provide a basis for differentiating between gaseous, liquid, and solid matter at the location of the capsule in the GI tract. Readings of the reflectometer enable the antenna 17 and directional coupler 171 to operate in cooperation as an environmental dielectric sensor.

The readings of the ingestible capsule 10, which include one or more from among readings from: the temperature sensor 14a, the heater side 132b of the VOC gas sensor 132, the sensor side 132a of the VOC gas sensor 132, and the TCD gas sensor 131, may also include readings of the reflectometer. Hence, change in capsule location within the GI tract causes a change in reflectometer readings, and therefore provide an indicator that a transition event between two sections of the GI tract has occurred.

The ingestible capsule 10 may further comprise an accelerometer 19. The accelerometer 19 may be a tri-axial accelerometer. A rate of change of angular position or orientation of the capsule 10 is somewhat dependent upon location within the GI tract, and therefore accelerometer readings provide an indicator that a transition event between two sections of the GI tract has occurred. The accelerometer readings may measure angular acceleration about three axes of rotation, wherein the three axes of rotation may be mutually orthogonal.

### Processor Hardware, Memory Hardware

The processor hardware and memory hardware may be separate components or may be part of the same single integrated chip. The processor hardware and memory hardware are selected according to the particular implementation requirements of each design or version of the capsule 10, noting that constraints such as power consumption, cost, data throughput, size of data transmission payload, etc, will vary between designs or versions. The processor hardware may be a processor or a plurality of interconnected processors.

### Pairing

The wireless data transmitter may be a Bluetooth transmitter, a wifi transmitter, a radio transmitter, or another form of wireless data transmitter. A radio transmitter may be configured to transmit in the 433 MHz band. In any case, the wireless data transmitter may be provided as part of a wireless data transceiver. For example, the wireless data transceiver may receive signals at least in performing pairing or any other form of coupling to a recipient device 30. The capsule 10 may be configured to enter into a wireless pairing or coupling mode immediately upon initiation (i.e. first power-on), wherein a subject or another user is instructed (via written instructions or via an application running on the recipient device itself) to pair or couple the capsule 10 to the recipient device 30 prior to ingestion of the capsule 10. However, embodiments may be configured such that pairing or coupling is not necessary, for example the capsule 10 may be configured to broadcast data to a recipient device in a data transmission technique that is agnostic to pairing or coupling status, as discussed in more detail below.

### Data Transmission Techniques

There are two principal data transmission techniques, which ingestible capsules may be configured to use either or both of, depending on implementation details (i.e. use case). In a post-excretion data transmission technique, signals from the sensors are received at the processor hardware 151 (utilising also the storage capabilities of the memory hardware 152) and processed on-board the capsule 10 in order to identify and record motility indicators (and optionally also other characteristics of the sensor output or sensor readings of interest or groups of sensor readings of interest) and the recorded motility indicators (and optionally also the other characteristics, metrics, and readings or groups of readings of interest, such as peak H2, area under a plot of H2 against time) are stored on the memory hardware 152 as a data transmission payload. Other characteristics and readings or groups of readings of interest may include, for example, maximum or minimum readings from specific sensors or from metrics calculated by combining sensors. The maximum or minimum readings may be local maximum or local minimum readings, wherein local is defined by, for example, predefined timings or motility events determined to have occurred by the capsule 10 itself. A specific example is maximum or minimum H2 concentration, which is a metric calculated from the gas sensor readings by an appropriately calibrated processor hardware. The data transmission payload is transmitted by the wireless transceiver once excretion of the capsule 10 from the GI tract is detected (for example by the temperature sensor 14a signal and/or by the accelerometer 19 signal). Metrics further include peak H2 level or value, timing of peak H2, and total H2 (area under the curve). Such metrics may be calculated by the on-board processor hardware 151 during passage through the GI tract of the subject, and transmitted away from the capsule 10 to a receiver device in post-excretion transmission as part of a report or otherwise.

In the post-excretion data transmission technique, the transmission may be via a Bluetooth transmission mode that is not dependent upon pairing status. That is, for example, if the Bluetooth transceiver is paired to a receiver device then it transmits the data transmission payload to the paired receiver device, and if the Bluetooth transceiver is unpaired then it broadcasts the data transmission payload to a recipient device in the absence of pairing in an inquiry mode (which may be referred to as discovery mode or beacon mode). Bluetooth protocol has an inquiry mode in which a device broadcasts a unique identifier, name and other information. The data transmission payload, or part thereof, may comprise or be included in the said other information. In particular, the data transmission payload may be prioritised or otherwise filtered by the processor hardware 151 so that information deemed particular important such as an indication that excretion has occurred (it is important for clinical reasons to know that the capsule 10 has been excreted) and potentially information such as timing of determined motility events, is transferred away from the capsule 10 in preference to other information. Following the inquiry mode transmission, the transceiver may again attempt to pair, connect, or otherwise couple, with the recipient device, and if successful, to transmit the remainder of the data transmission payload. Of course, said pairing, connecting, or coupling, may have been performed initially pre-ingestion so that post-excretion the Bluetooth transceiver is attempting to re-pair, re-connect, or re-couple, with the receiver device 30. It is noted that the present discussion uses Bluetooth as an example of a transmission protocol, but that the same techniques could be applied to different transmission protocols.

In the event that there is data transmission payload pending transmission away from the capsule 10 after the broadcast of the unique identifier, name, and other information during the Bluetooth inquiry mode, then capsule 10 may be configured to initiate or re-initiate a data communication connection (i.e. a pairing or re-pairing) with a receiver device 30. Upon successful initiation or re-initiation of the communication connection, transmission of the said data transmission payload pending transmission away from the capsule 10 is performed whilst the data communication connection remains active.

The Bluetooth transceiver 18, or any other wireless data transmitter 18, may be configured to automatically re-connect following an initial (i.e. pre-ingestion) connection to a receiver device 30. The receiver device 30 may run an app or web app to guide the subject in terms of how to ingest the capsule 10, to notify the subject that the excretion event has been determined, and optionally also that the data transmission payload has been successfully transmitted to the receiver device 30 and so the capsule 10 may be flushed away. It is noted that the terms pair, connect, and couple, are interchangeable in the present document, each representing the establishment of a wireless connection between two devices for wireless data transfer.

It is noted that data transmission payload may be being transmitted throughout passage of the capsule 10 through the GI tract, dependent upon pairing, coupling, or connection to the receiver device 30. However, confirmation that occurrence of an excretion event has been determined by the capsule is information that is of particular importance since safety of capsule 10 is reliant on the capsule 10 being excreted. Therefore, information representing determination of occurrence of the excretion event (i.e. a report thereof) is prioritised and may be transmitted in a broadcast or inquiry mode, whereas the remaining data transmission payload is transmitted once connection between the wireless data transmitter 18 and the receiver device 30 is established.

In Bluetooth inquiry mode, data can be transmitted to the receiver apparatus 30, or to any Bluetooth receiver apparatus within range of the capsule 10, without pairing. The wireless transceiver 18 is operable in a Bluetooth inquiry mode or a Bluetooth low energy mode. Capsules 10 may store and transmit among the data transmission payload readings from one or more sensors representing a predefined period either side of the identified motility indicators. For example, gas sensor signals only, or for all sensors. Such readings may be used to add confidence to the identified motility indicators in terms of determining whether or not a motility event has occurred, and/or may provide other information useful in a health or clinical context.

More generally, data transmitted according to the post-excretion data transmission technique may be any of the data transmission payload that has not already been transmitted. For example, the wireless data transmitter 18 may be configured to transmit the data transmission payload to a paired receiver apparatus while still in the GI tract (this transmission is referred to herein as pre-excretion data transmission technique). However, owing to issues such as signal attenuation, noise, power supply issues, temporary pairing failure, or if pairing was never performed in the first place, or for any other reason, some or all of the data transmission payload may be pending transmission at the point of excretion. In that case, the remaining data transmission payload is transmitted according to the post-excretion data transmission technique once excretion is detected. It is noted that down-sampling of the data transmission payload may be performed prior to transmission via the post-excretion data transmission technique. Furthermore it is noted that some elements of the data transmission payload may be prevented from transmission via the post-excretion data transmission technique. For example, since bandwidth, and also time within which to transmit, may be limited, it may be that the motility event indicators and diagnostic indicators themselves are included, but that sensor readings are excluded from the data to be transmitted according to the post-excretion data transmission technique.

In a pre-excretion data transmission technique, the sensor signals are transmitted continuously by the wireless transceiver 18. In the pre-excretion data transmission technique, the process hardware 151 coordinates the receipt of the signals from the sensors and the storage at the memory hardware 152 for transmission by the wireless transceiver 18.

In the example of a Bluetooth wireless transceiver 18, in the pre-excretion transmission technique the transceiver may be operated according to a long-range or coded-phy Bluetooth transmission procedure, such as BTLE Coded PHY. A signal power enhancement of around 10 dB is achievable via BTLE Coded PHY Bluetooth transmission procedure.

During a data transmission phase of the ingestible capsule 10 (i.e. which in the post-excretion data transmission technique is in a short burst post-excretion and in the pre-excretion data transmission technique is continuous while the ingestible capsule 10 is in use, that is, in the GI tract of a subject mammal 40 and obtaining and transmitting readings) the wireless transmitter 18 transmits the readings to a receiver apparatus 30, which may be a dedicated device for receiving and storing the readings (and optionally with a user interface) or may be a multi-function device such as a mobile phone (such as a smart phone). The mobile phone may be running an application which processes some or all of the data transmission payload to generate a motility report or diagnosis of a medical condition based on motility indicators and diagnostic indicators either included in the data transmission payload or derivable therefrom. Alternatively, the application may be configured to transmit the data transmission payload on to a server or another processing apparatus to generate the motility report or diagnosis based on the data transmission payload. The subject mammal need not remain within a specific range of the remote computer 20 during the live phase. Capsules 10 equipped with a Bluetooth transceiver 18 may communicate directly with a smartphone of a user, which obviates any need for a dedicated receiver apparatus (the smartphone taking on the role of receiver apparatus 30). The receiver apparatus 30 (whether a dedicated device or a mobile phone or tablet computer) may process the readings itself or may upload the readings to a remote computer 20 for processing (i.e. identifying motility indicators, determining motility event timings, resolving gas analytes). The upload may be continuous during a live phase of the capsule, or the upload may be after the live phase of the capsule is terminated. The receiver apparatus 30 may also store the readings, so that loss of connectivity between the receiver apparatus 30 and a remote processing apparatus is not critical.

The on-board processor 151 may apply one or more processing or pre-processing steps, as discussed in more detail below. Digitisation of the readings is performed either by the sensors themselves, by the processor 151 or by the wireless transceiver 18. The digitised readings are transmitted via the antenna 17. The readings of the capsule 10 are made at an instant in time and are associated with the instant in time at which they are made. For example, a time stamp may be associated with the readings by the microcontroller 15, the wireless transmitter 18, or at the receiver apparatus 30 or remote computer 20. For example, if readings are made and transmitted more-or-less instantaneously (i.e. within one second or a few seconds) by the wireless transmitter 18 then the time of receipt by the receiver apparatus may be associated with the readings as a time stamp. Processing of the readings discussed further below is somewhat dependent on the relative timings of the readings (i.e. so that contemporaneous readings from the different sensors can be identified as contemporaneous), however accuracy to the level of one second, a few seconds, or 10 seconds, is sufficient.

In a hybrid mode, capsules 10 may combine the two data transmission techniques. For example, the capsule 10 may process sensor readings on-board to identify motility markers (and optionally also other readings or groups of readings of interest) for transmission in Bluetooth inquiry mode immediately post-excretion. In addition, the capsule 10 may continuously transmit sensor readings to a paired receiver apparatus. Optionally, the continuous transmission may be of the gas sensor signals only, or gas sensor signals and temperature sensor signals required to calibrate gas sensor signals. Gas sensor signals are of particular interest in providing health and clinical information, particularly once combined with motility indicators provided by the other sensors such as accelerometer, reflectometer. Gas sensor signals may be downsampled or subject to other compression techniques by the on-board processor prior to transmission. Optionally, the on-board processor hardware 151 may apply one or more filters, such as a high pass or low pass filter applied to the values themselves or to the derivative with respect to time, so that only gas sensor signals meeting particular thresholds are included in the data transmission payload. Metrics representing gas sensor signals, such as peak of a derived H2 value, or area under a plot of derived H2 value with respect to time, may be maintained and transmitted away from the capsule 10.

For capsules 10 configured to perform data transmission during passage through the GI tract (i.e. pre-excretion data transmission technique), commercial bands (such as 433 MHz) are used by the antenna 17 as electromagnetic waves in this frequency range can safely penetrate the mammalian tissues 40. Bluetooth may also be used in such capsules, wherein Bluetooth may be long-range Bluetooth, particularly when BMI of the subject (human) is above a threshold, or a high level of attenuation is expected for some other reason. Other commercial bands and protocols may be used in various applications, such as LoRa. Coding may be applied at the digitisation stage to assure that the data transmitted by the capsule 10 is distinguishable from data transmitted by other similar capsules 10. The transmission antenna 17 may be, for example, a pseudo patch type for transmitting data to the outside of the body data acquisition system.

Power source 16 is a battery or super capacitor that can supply the power for the sensors and electronic circuits including the processor hardware 151 and memory hardware 152. A life time of at least 48 hours may be set as a minimum requirement for digestive tract capsules. A number of silver oxide batteries in the power source 16 is configurable, depending on the needed life time and other specifications for the capsule. For example, long-range Bluetooth may consume more power than standard Bluetooth. Capsules may be configured to switch from long-range Bluetooth transmission to standard Bluetooth transmission once the stored energy in the battery (or batteries) drops below a predefined threshold, wherein the on-board processor is configured to monitor stored energy level.

### Data Processing Approaches

In broad terms, the processing of the signals/readings/data may include any combination of three main aims: firstly (first branch or motility branch) to assess capsule motility through the GI tract by determining timing of motility events including ingestion, gastric emptying (gastric-duodenal transition), ileocecal junction (ICJ) transit/transition, and excretion (and thus to generate report data representing capsule motility and/or to change internal capsule settings such as data transmission settings in response to determination of occurrence of motility events). The second aim (second branch or gas resolution branch) is to determine constituent gases and the concentrations thereof in the gas mixture at the location of the ingestible capsule 10 throughout or at one or more points of its journey through the GI tract. The third aim is to diagnose medical conditions by detecting or identifying predefined diagnostic indicators in the readings of the sensor hardware, wherein the diagnostic indicators are characteristic features of the signal output by an individual sensor, a combination of sensors, or a metric calculated by processing the signal output by a single sensor or a combination of sensors. Diagnostic indicators are predefined based on specific trials and associated research. Conditions that may be diagnosed in this way include, for example, small intestinal bacterial overgrowth (SIBO), constipation, and gastroparesis. Capsules 10 disclosed herein are at least concerned with the first main aim, noting that a particular benefit of accurately determining the location of the ingestible capsule 10 in the GI tract is to provide context to the determinations of constituent gases (i.e. gas analytes) and their concentrations. However, it is noted that the outcomes of the first branch of processing provide useful information in the assessment of gut health even in the absence of the second branch of processing, and may have other utility beyond the second branch of processing. Optionally, determinations of the second branch of processing may be utilised to add confidence to determinations in the first branch of processing. Furthermore, it is noted that in any case, it is desirable to report to the receiver apparatus that the occurrence of the excretion motility event has been determined, since confirmation of excretion notifies subject and clinician that the capsule 10 no longer resides within the GI tract.

Readings from different sensors or pseudo sensors (wherein the reflectometer may be referred to as a sensor or as a pseudo sensor) will be used in the first branch and/or the second branch and/or third branch as appropriate. For example, the TCD gas sensor readings are utilised for detecting a gastric duodenal transition indicator in the first branch, and in the second branch for, for example, determining concentration of H2 at the location of the capsule 10. The readings from the VOC heater side are used in the second branch as a hotter TCD sensor, to increase the temperature range at which TCD readings are obtained and thus to increase the range of H2 concentrations that are detectable. The VOC sensor side is sensitive to both O2 and H2 as well as other gases and so these readings may be utilised in the second branch. Other gases include CH4 and SCFAs. Optionally, the VOC sensor side readings are not used in the second branch and the VOC sensor side readings are only used to detect an ileocecal junction transition indicator. Optionally, the VOC sensor side (i.e. the VOC sensing element) forms a resistor in a voltage divider network, the output of which is measured as the VOC sensor side reading. A transform may be applied at the capsule 10 and/or as part of the processing to transform the output of the voltage divider network into a resistance measurement from the sensing element. The VOC sensor side may be driven with a consistent (i.e. repeated) voltage pulse profile. VOC sensor side readings may be taken in sync with the voltage pulse profile so that there is no phase shift between the voltage pulse and the timing of the readings. CH4 concentration is determined from the TCD gas sensor readings and/or from the VOC heater side readings.

The on-board sensors generate a large amount of data. Limitations such as energy capacity of power source 16 mean that it may be preferable to process some data on-board the capsule 10 in order to extract a (relatively smaller) data transmission payload from the (relatively larger) generated data. In addition to extraction, data processing techniques may summarise or otherwise represent the generated data in order to reduce the size of the data transmission payload. The processor hardware 151 may be configured to prioritise contents of the data transmission payload. In particular, data representing that the excretion event has been determined and the timing thereof may be given highest priority (i.e. transmitted in preference to other content of the data transmission payload pending transmission at the same time as the data representing that the excretion event is pending transmission).

It will be appreciated that there is a full spectrum of possibilities between, at one extreme, transmitting all generated data away from the capsule 10 for processing elsewhere (i.e. from capsule perspective a high data transmission burden and low data processing burden) and at the other extreme performing a high degree of processing on board to determine results including timings of motility events to a high degree of certainty and even to diagnose specific health conditions or ailments, and only transmitting the said processing results (i.e. from capsule perspective a low data transmission burden and high data processing burden).

Embodiments are configurable at the design stage according to implementation requirements to combine data processing and data transmission in a manner that enables data processing to occur, whether on-board or at a receiving apparatus 30 or remote data processing apparatus 20, to determine motility events, and other gut health indicators such as gas constituent concentrations at one or more locations/timings in the GI tract, and to identify or detect diagnostic indicators.

It is noted that the data transmission techniques detailed above may be considered orthogonal to the data processing approaches, in the sense that which data transmission technique, or combination of data transmission techniques, is selected does not necessarily dictate the data processing approach. However, of course, the data transmission capacity of each technique must be considered in deciding how much processing to perform on-board the capsule 10, noting that, in general, processing on-board the capsule 10 reduces the size of the data transmission payload, on the assumption that processing results are included in the data transmission payload in place of readings processed to generate said processing results.

In an example in which data transmission capacity is large, for example because data is to be transmitted according to the pre-excretion data transmission technique or according to both the pre- and post-excretion data transmission techniques, the raw sensor signals may be transmitted to the receiver apparatus 30 for processing off-board (i.e. not on the capsule). The receiver apparatus 30 or another processing apparatus 20 connectable thereto (for example via a wired or wireless data transmission connection) may process the signals to identify motility indicators, determine motility event timings, and resolve analyte gases from gas sensor signals. Capsules 10 may transmit data using a Bluetooth long range mode (coded PHY).

Some processing of the sensor readings may still be performed on-board the capsule 10, for example to identify or detect motility event indicators, or diagnostic indicators, with the processing result added to the data transmission payload for transmission (in the first instance) according to the pre-excretion data transmission technique, and with the post-excretion data transmission technique being used as a fallback in case the data transmission payload is not successfully transmitted pre-excretion.

Further examples of on-board processing of the sensor readings includes, for example, processing a signal from the accelerometer to calculate a metric representing capsule agitation or overall capsule motion. The calculated metric may be calculated periodically based on the accelerometer signal from the preceding period (for example every 1 minute, 2 minutes, 3 minutes, 5 minutes, 10 minutes), and the calculated metric per period time stamped and added to the data transmission payload.

The term signal may refer to the output signal produced by a sensor, whereas the term reading may refer to a specific measurement of the signal taken at or otherwise associated with an instant in time, which instant in time may be included with or associated with the reading explicitly or implicitly (i.e. if the reading is the 1000^{th} reading in a series and readings are taken at a rate of 1 Hz and the timing of the first reading in the series is known, then the position of the reading in the series implicitly represents the timing). Time stamps or other timing indicators may be provided by the processor hardware 151.

On-board processing may be performed in more-or-less real time, allowing for latency caused by transfer between components and processing itself. Alternatively, the readings may be received by a receiver apparatus 30 processed thereby and/or stored for upload and processing retrospectively by a remote processing apparatus 20. Such retrospective processing may be performed by analysing the most recent readings first (i.e. in reverse chronological order), so that the first event timing to be determined is excretion, followed by ICJ, then GET, then ingestion. Or the analysis may be of the readings in chronological order. Other dependencies may exist between indicators or markers in the data which constrain an order in which readings are processed.

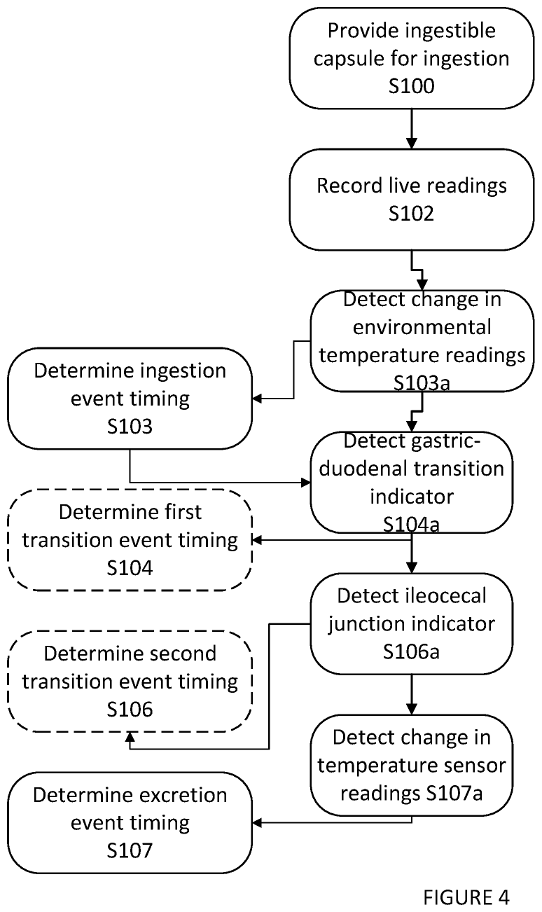

FIG. 4 represents an exemplary flow of processing tasks and exchange of data in an example of the post-excretion data transmission technique, in which signals from the sensors are received at the on-board processor hardware 151 (utilising also the storage capabilities of the memory hardware 152) and processed on-board the capsule 10 in order to identify and record motility indicators (and optionally also other characteristics of the sensor output or sensor readings of interest or groups of sensor readings of interest) at steps S100, S104a, S016a, and S107a and the recorded motility indicators (and optionally also the other characteristics and readings or groups of readings of interest) are stored on the memory hardware 152 as a data transmission payload.

In the example of FIG. 4, the suffix a in the reference signs S103a, S104a, S106a, and S107a represents the detection or identification of an indicator, whereas the corresponding numeral without the suffix represents the determination that an event indicated by the correspondingly numbered indicator has occurred. Excretion event timing is determined at S107, at which point the capsule 10, or specifically the Bluetooth transceiver 18, enters beacon transmission mode, and the data representing the detected motility indicators from steps S104a and S106a is transmitted to the receiver apparatus 30. At the receiver apparatus 30 or a remote computer 20 in data communication therewith, the motility indicators from steps S104a and S106a are processed to determine first transition event timing (gastric-duodenal transition) at S104 and second transition event timing at S106. Steps S104 and S106 are illustrated in dashed lines to indicate that they are not performed on-board the capsule, but are performed at the receiver apparatus or a remote computer connected thereto. The determinations may be part of the same processing as the detections, depending on the level of confidence required and whether or not there exists potential for false positives.

In the example above, a distinction is made between the on-board processing required to identify or detect a motility event indicator is treated as distinct from the off-board processing to analyse the indicator and optionally also contemporaneous readings from other sensors or pseudo sensors to determine whether or not the indicator is caused by a motility event. For example, by calculating a confidence level and comparing the confidence level with a threshold. However, it is noted that said determination may be performed on-board the capsule. In particular, there may be on-board processing applied to the detected motility indicator (for example, calculate an extent of a signal spike or a signal gradient) to characterise the motility indicator, compare the characterisation with a predefined threshold, and if met, to determine that the motility event caused the motility indicator.

It is noted that should the capsule 10 be configured to operate in accordance with the pre-excretion data transmission technique, the sensor signals are transmitted continuously by the wireless transceiver 18, so steps S103a, S103, S104a, S104, S106a, S106, S107a, and S107, may all be performed off-capsule in that case, either by the receiver apparatus 30 or by a remote processing apparatus 20 in data communication with the receiver apparatus 30.

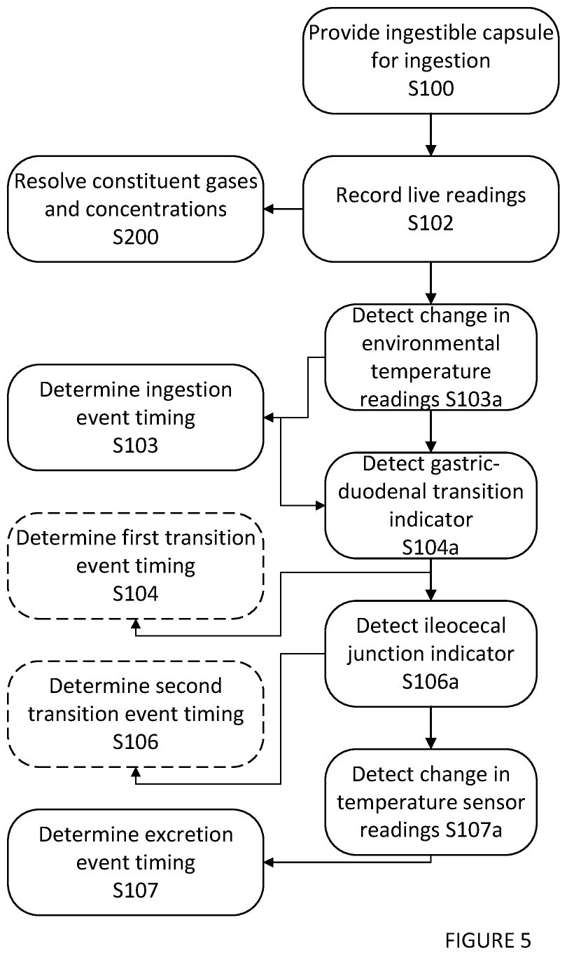

A variant of the method of FIG. 4 is illustrated in FIG. 5, in which some recorded readings from S102, in particular the gas sensor readings, are transferred from the capsule 10 to the receiver apparatus 30 (and optionally on to a remote processing apparatus 20) for processing to resolve constituent gases of the gas mixture at the capsule during its journey through the GI tract. The gas sensor readings may be transmitted continuously (in accordance with the pre-excretion data transmission technique) or may be transmitted during beacon mode transmission (i.e. in accordance with the post-excretion data transmission technique) following detection of the excretion event at S107. The gas sensor readings that are transmitted for processing in S200 may be for the entirety of the GI tract passage or may be for a predefined time period either side of one or more of the detected indicators, in particular gas sensor readings representing a predefined period (such as 1 minute, 2 minutes, 5 minutes, 10 minutes, 15 minutes, or 30 minutes) either side of the timing of the detected gastric-duodenal transition indicator S104a and/or the detected ileocecal junction transition indicator S106a. For example, the gas sensor readings from said time periods may be helpful in adding confidence to the detected motility indicators and determining whether or not the indicators are caused by motility events of the capsule through the GI tract. Furthermore, the gas sensor readings from those time periods may be of particular interest in detecting health conditions and assessing condition and health of the GI tract.

### Monitoring for Excretion Event

Steps S107a and S107 are performed on-board the capsule by the on-board processor 151. If the temperature sensor readings being monitored for a change at S107a that is determined to be caused by an excretion event at S107 include temperature readings, then a potential for false positives exists insofar as the capsule may experience a temperature drop in the stomach due to consumption of a cold drink or another cold foodstuff by the subject. Such false positives may be avoided by the following techniques:
In a first technique the ingestion event timing is determined on-board the capsule at S103 and the determined ingestion event starts a timer. The timer may be, for example, six hours. Step S107a, processing temperature sensor readings to detect a change that may be caused by excretion, does not begin until after expiry of the timer. Thus, other than in very unusual circumstance, the capsule 10 has progressed past the stomach and a temperature drop or other such change in temperature sensor readings can be attributed to excretion rather than consumption of a cold drink or foodstuff. The first technique presents difficulties since time may not be an accurate predictor of capsule progress, particularly in the case of gastroparetic patients.
In a second technique the detection of the ileocecal junction transition indicator at S106a and the determination of the ileocecal junction transition event S106 are both performed on-board the capsule by the processor 151. Step S107a, processing temperature sensor readings to detect a change that may be caused by excretion, does not begin until after determination that the ileocecal junction transition event has occurred. In a variation of the second technique, gastric emptying (i.e. passage of the capsule 10 out of the stomach) could be detected on-board the capsule and used as an early bound of the excretion monitoring at S107a.
The third technique is a variant of the second technique in which, for example, it may be that the detection of an ileocecal junction transition indicator at S106a, regardless of whether the determination S106 is performed on-board or off-board, is the trigger to begin S107a, processing temperature sensor readings to detect a change that may be caused by excretion. A variation of the third technique is to use another means to determine that the capsule 10 is present in the small intestine or large intestine to trigger the start of monitoring step S107a. For example, in capsules including an accelerometer 19, detected orientation changes or a metric representing orientation changes may indicate travelling through the intestines. Similarly, in capsules including a reflectometer, the reflectometer signal being within a predefined range may be used to detect presence in the intestines and thus to initiate monitoring step S107a. In a further example in which gas sensor signals are processed to calculate concentrations of constituent gases in the gas mixture entering the capsule headspace, it might be that calculated H2 levels are in a predefined range only expected in the large bowel, thus H2 levels entering said range is a trigger to being monitoring step S107a. In another example, the gas sensors may include a VOC gas sensor or some other means to sense VOC concentration, wherein the sensed VOC concentration being within a particular range is taken as a trigger to being monitoring step S107a.

### Detecting Indicators and Determining Events

References to an initiation event refer to: a power on event of the capsule initiating a live phase during which the capsule is active and readings are generated by the sensors and received by the receiver apparatus; or an initiation of recording by a button press on a user interface of the receiver apparatus 30 (so that it is possible that the capsule is already powered). The live phase refers to the time during which the capsule is powered on and readings are being recorded (i.e. stored or relayed) by the receiver apparatus 30.

References to a termination event refer to an end of the live phase, which termination event may be: a power off event of the capsule ending the live phase; or a termination of the live phase by a button press on a user interface of the receiver apparatus 30.

At S100 the ingestible capsule 10 is provided to the subject mammal 40 for ingestion. The ingestible capsule 10 is as illustrated in any of FIGS. 1A, 1B, 2, and comprises, inter alia, a housing 11, a power source 16, processor hardware 151, memory hardware 152, a temperature sensor 14, a TCD gas sensor 131, and a VOC gas sensor 132. The ingestible capsule 10 may be stored in a powered down state in contact with packaging, wherein separating the ingestible capsule 10 from the packaging causes the powered down state to end and the capsule 10 to enter a powered state. Entering of the powered state may be the initiation event, or the initiation event may require the capsule to enter the powered state and a button press (or other interaction) with a user interface on a receiver apparatus 30. It may be that separation of the capsule 10 from the packaging is the event that causes the capsule to be powered on and the obtaining and processing of readings by the capsule 10 to begin.

It is expected that ingestion will take place soon after initiation. Ingestion may be sensed by on-board sensors and detected by processing the readings thereof, or may be expressly indicated by an interaction of the subject 40 with a user interface on a receiver apparatus 30.

At S102 recording of the readings begins. Recording means storing for downstream processing, and does not mean or imply permanent storage. Certain readings may be retained or discarded following processing, according to the configuration of the embodiment. The readings are recorded by the capsule 10, for example at the memory hardware 151. The readings include readings of the TCD gas sensor 131, readings of the sensor-side of the VOC gas sensor 132a, and may also include one or more from among temperature sensor readings, readings from the heater side of the VOC sensor 132b, readings from the reflectometer (i.e. the antenna 17 and directional coupler 171), and readings from the accelerometer 19. The readings may be recorded as a function of time, or time may be derivable from a position within a sequence. The temporal value assigned to each reading may be assigned at the capsule 10, for example by the microcontroller and/or the wireless transmitter 18, may be assigned by a receiver apparatus 30 based on a time of receipt of the respective readings from the capsule 10, and/or may be assigned by a remote computer 20 based on time of receipt from the capsule 10 or from the receiver apparatus 30. Alternatively or additionally a temporal value assigned to each reading may be based on order of arrival. For example, if it is known that TCD gas sensor readings are obtained every n seconds, then the mth reading is timed at m×n seconds (or m−1×n, depending on implementation) after the initiation event starting the live phase. It is noted that temporal values may be relative to a baseline such as the capsule 10 entering a powered state rather than being absolute values of time based on a calendar and time of day value.

The steps are illustrated in a serial manner in FIGS. 4 & 5, but in practice the obtaining and recording the readings S102 may be performed whilst the processing steps S103 to S107 are being performed. Optionally, some processing may be performed after the recording of the readings S102 has been completed and the capsule excreted. As discussed above, processing of sensor signals may be performed on-board, at a receiver apparatus such as a smartphone, or on a computer in data communication with the receiver apparatus. The processing may be performed on the cloud. The processing may be performed on server computing apparatus connectable to the capsule 10 via an internet connection to a receiver apparatus 30. The receiver apparatus itself may perform some or all of the processing steps S103 to S107.

Each determination step: determining ingestion event timing S103; determining first transition event timing S104; determining second transition event timing S106; determining excretion event timing S107; has a respective associated detection step. In general, the detecting steps comprise processing and analysing the recorded readings to identify indicators (i.e. markers) that indicate an event associated with the motility of the capsule 10 may have occurred. The respective determining step, in addition to the detecting, includes applying a condition or some other logic to the detected indicator to determine (to within a confidence level) that the indicator was caused by a motility event, and thus the motility event can be determined to have occurred at (or around) the timing of the detected indicator. Motility events include one or more from among the ingestion event, the gastric-duodenal transition, the ileocecal junction transition, and the excretion event. Gastrointestinal motility is defined by the movements of the digestive system, and the transit of the contents within it. The indicator is a feature in a plot of recorded readings vs time from the relevant sensor or pseudo sensor. The feature is a step, bump, inflexion point, or gradient change. Particular indicators may be more specific, for example the condition may be more specific than the indicator simply being a step, bump, inflexion point or gradient change. Embodiments may combine the detecting and determining steps into a single processing thread or processing event that achieves determination.

Indicators may be detected in readings from a first sensor. An indicator is associated with a hypothesis that the indicator was caused by an event associated with the motility of the capsule. Confidence may be added to the hypothesis by obtaining readings from other sensors at the timing of the indicator (and around said timing) and detecting confirmatory indicators in those readings. For example, hydrogen (H2) levels vary through the GI tract and so readings of H2 levels may be used to add confidence to readings from other sensors. Readings of H2 levels may be used as a basis for an ileocecal junction transition indicator at S106a. H2 levels may be sensed directly or may be derived, such as derived from TCD gas sensor readings. In particular, an ileocecal junction transition indicator may be detected by identifying an increase in (sensor side) VOC gas sensor output exceeding a predefined threshold with a contemporaneous, or temporally adjacent to within a predefined temporal distance either side, increase in H2 levels exceeding a predefined threshold. Noting that H2 levels are determined from the TCD gas sensor output and/or heater-side VOC sensor output.

Similarly, readings of CH4 levels may be used as a basis for an ileocecal junction transition indicator. In particular, an ileocecal junction transition indicator may be detected by identifying an increase in (sensor side) VOC gas sensor output exceeding a predefined threshold with a contemporaneous, or temporally adjacent to within a predefined temporal distance either side, increase in CH4 levels exceeding a predefined threshold. Noting that CH4 levels may be determined from the TCD gas sensor output and/or heater-side VOC sensor output.

Embodiments may be configured to perform ileocecal junction transition indicator detection S106a on-board the capsule, since detection thereof acts as a trigger to begin monitoring temperature sensor readings to detect the change therein at S107a.

Different subsets of the recorded readings may be analysed in order to detect different indicators. The subsets may be partitioned according to timing and according to the sensor from which they were obtained. For example, monitoring signals to detect a particular indicator may be triggered by detection of a preceding indicator. Since indicators have a defined order an upper and/or lower bound on timing of a particular indicator may be provided by determined timings of directly adjacent indicator(s) among the defined order.

It is noted that the term sensor is used broadly to encompass not only the sensors per se (i.e. the TCD gas sensor 131, the sensor side of the VOC gas sensor 132a, and optionally the environmental sensor 14 and/or the accelerometer 19), but also the components that provide readings and are not sensors per se, such as the directional coupler 171 and the heater side of the VOC sensor 132b (which components may be referred to as pseudo sensors). The term sensor encompasses the sensors per se and the pseudo sensors.

At S103a the recorded readings from the temperature sensor 14a are analysed to detect a change in the environment that would indicate an ingestion event. In this context, the change may be a change in environmental temperature indicated by the readings of the environmental temperature sensor 14a, or the change may be a change in environmental humidity indicated by the readings of the environmental humidity sensor 14b combined with the readings of the temperature sensor 14a. The detection may be based on readings from both the environmental temperature sensor 14a and the environmental humidity sensor 14b, either to add confidence to one another, or to account for unusual ambient humidity or temperature conditions which could reduce the change in one condition of the other upon ingestion (i.e. ingestion on a hot day may not register a significant temperature change, but would, in many circumstances, register a significant humidity change). In on-board processing, the analysis may be of environmental sensor readings from an initiation event (such as power on of the capsule 10) forwards, with an end to S103a being set by determination of the ingestion event timing S103. That is, once it is determined that a detected change in the environmental sensor readings is caused by an ingestion event, no further processing to detect an ingestion event is performed. Detecting the change at S103a may be on a rolling basis by comparing a subject one or more readings with a predetermined number of preceding readings, with a difference of more than a threshold (i.e. one or two degrees centigrade or one or two % relative humidity) being a detected change. Determining the ingestion event timing may include comparing the temperature or humidity of the subject reading with an expected temperature or humidity for the environment at the start of the GI tract of the subject mammal 40, wherein being within a threshold is a determination that the capsule 10 has been ingested. Alternatively the condition may be that a predefined number or more consecutive readings are within a threshold of the expected temperature or humidity for the environment at the start of the GI tract of the subject mammal.

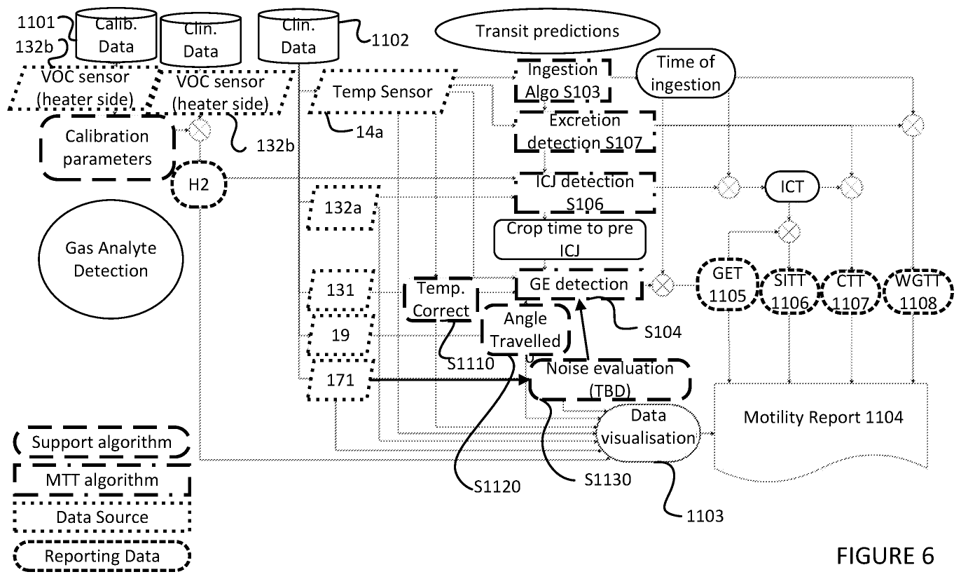

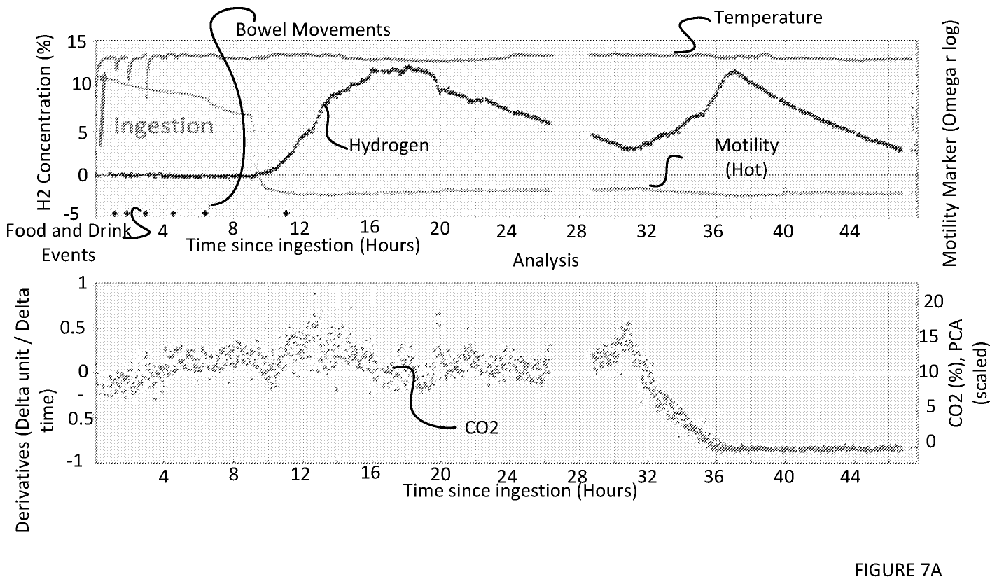

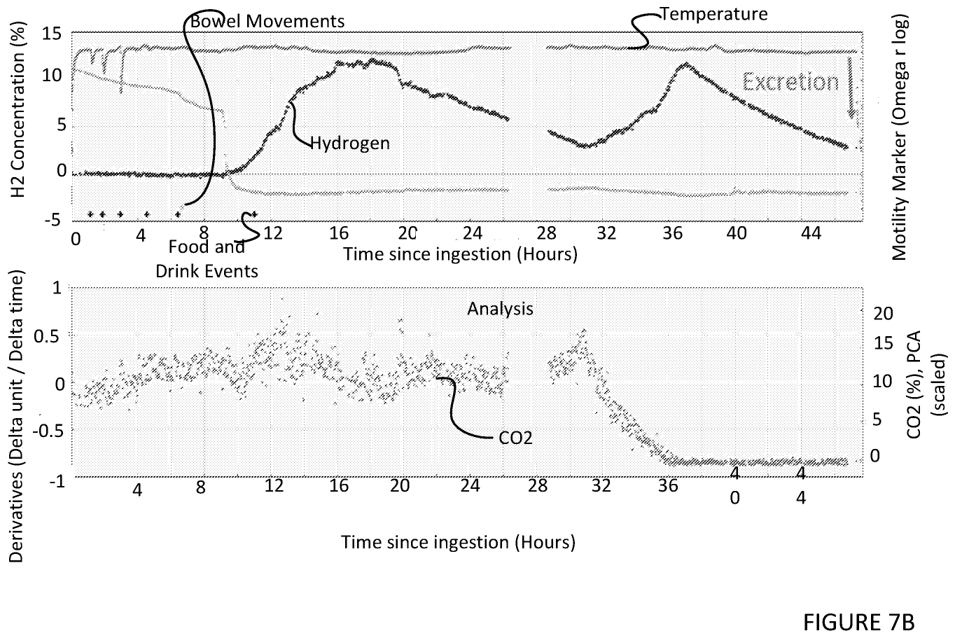

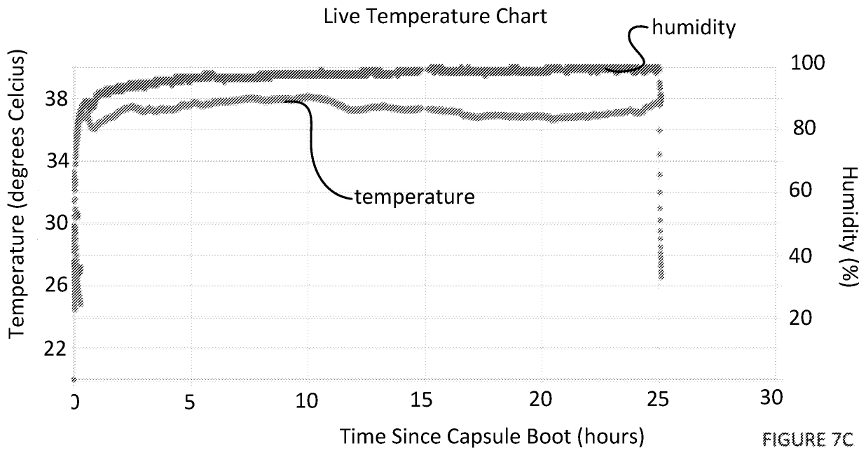

FIG. 6 illustrates exemplary relationships between sensors, algorithms, and processing results in an embodiment. Calibration data 1101 is lookup tables etc for calibrating the VOC sensor for operating as a TCD sensor in different environmental temperatures, which combines with the heater side of the VOC sensor 132b to provide calibration parameters. Clinical data 1102 is the knowledge that changes in VOC sensor heater side readings are associated with a change in H2 concentration in subject gas mixture, which feeds into ICJ detection at S106 & S106a, and is in itself an output data entity at 1103. Like reference numerals are used for equivalent features in other Figures, and so a full description of the features of FIG. 6 is disclosed herein by reference to the other Figures. Noting that the ingestion algorithm executes steps S103 & S103a, the excretion detection algorithm executes steps S107 & S107a, the ICJ detection algorithm executes steps S106 & S106a, and the gastric emptying algorithm executes steps S104 & S104a. Said algorithms may be executed on-board the processor, at a coupled receiving device, or at a remote computing apparatus connected thereto. Step S1110 is the correction of the TCD sensor readings to account for changes in environmental temperature. Step S1120 is applying an algorithm to process the accelerometer data as described below in relation to the first, or angle travelled, technique. S1130 is an exemplary processing algorithm for the reflectometer data and is determining changes in noise in the output signal thereof. Other processing algorithms may be applied to the accelerometer and reflectometer data. Algorithms S1110, S1120, & S1130, may be considered to be pre-processing algorithms, and may be performed on-board the capsule 10 to constrain the amount of data in the data transmission payload. The event timings determined by the algorithms are combined with one another to determine event timings of one or more from among: ingestion, gastric emptying, ileocecal junction transition, and excretion. The event timings are in turn combined to determine transit time metrics including gastric emptying timing 1105, small bowel transit time 1106, colon transit time 1107, and whole gut transit time 1108. These are included in an output motility report 1104. A data visualisation 1103 is such as illustrated in FIGS. 7A, 7B, 7C, 8, 9A, & 9B, for example. GET 1105 is gastric emptying time, SITT 1106 is small intestine transit time, CTT 1107 is colon transit time, and WGTT 1108 is whole gut transit time. MTT algorithm is motility transit time algorithm.

FIGS. 7A & 7B illustrate plots of capsule readings vs time since initiation event (boot) for an ingestible capsule 10 which is ingested by a subject human, makes its way through the GI tract, and is then excreted. The ingestion events and excretion events are marked. In the example, the external temperature is well below the internal temperature of the subject human. The plots also show hydrogen readings, motility readings, and CO2 readings, and are marked with food and drink events and bowel movement events (which events may be detected automatically or manually reported). The specific timing assigned to the ingestion event and the excretion event can be determined in a number of ways. FIG. 7C shows environmental temperature sensor readings and environmental humidity sensor readings vs time since initiation event (boot) for an ingestible capsule 10 which is ingested by a subject human, makes its way through the GI tract, and is then excreted. The events are not marked since it is evident from FIG. 7A where the ingestion and excretion indicators are detectable in FIG. 7C. The specific timing assigned to the ingestion event and the excretion event can be determined in a number of ways, noting that processing of readings may be performed on-board or off-board the capsule 10.

An example for the ingestion event: on a progressive (i.e. rolling) basis from a starting point being initiation event (in chronological case), determine mean value of three adjacent environmental sensor readings, determine a timing at which the mean value either began or ceased to be within a threshold distance of the expected environmental value (i.e. within 1 degree centigrade of expected temperature or within 1, 2, 5, or 10% of expected humidity) post-ingestion (i.e. average internal environment for subject), then determine that the ingestion event timing is the during the three readings (for example, the mid-point, the earliest point, or the latest point). The number three is exemplary and different numbers for the number of samples in the rolling average could be selected, such as five, ten, twelve, or twenty. Furthermore, the tolerance of 1 degree centigrade is configurable and could be, for example, 2 degrees, 3 degrees etc.

In the above example, ingestion event timing is determined by detection of an ingestion indicator (rise in environmental temperature readings) in the readings of the temperature sensor. The ingestion event timing is contemporaneous with the ingestion indicator. An ingestion indicator (i.e. marker) may be detected in the antenna reflectance signal from the directional coupler, the indicator being a step change in the readings (this is specific to embodiments in which the antenna 17 and directional coupler 19 operate as a reflectometer from which readings are taken). The ingestion event timing is contemporaneous with the ingestion indicator in the readings of the reflectometer. As a further example, the capsule may include a relative humidity sensor 14b as a form of environmental sensor 14, wherein an ingestion indicator may be detected by processing readings from said relative humidity sensor 14b. The indicator is the earliest (post-initiation event) rise of relative humidity to within a predefined threshold of 100%, for example, plus minus 5%, or plus minus 1%. A further ingestion indicator is a button press of an ingestion confirmation button on a user interface of a user device such as receiver apparatus 30 (whether that is a smartphone or a dedicated device for the present purpose). Embodiments may combine one or more of the disclosed ingestion indicators to determine ingestion event timing. For example, more than one of the disclosed ingestion indicators being detected at timings within a predefined timing window of one another, for example, one minute of each other, results in determination of ingestion event timing.

An example for the excretion event: on a progressive (i.e. rolling) basis from a starting point being ICJ event determination, determine mean value of three adjacent temperature sensor readings, determine a timing at which the mean value either ceased to be or began to be within a threshold distance of the expected environmental value (i.e. within 1 degree centigrade of expected temperature) pre-excretion, then determine that the excretion event timing is during the three readings (for example, the mid-point, the earliest point, or the latest point). The number three is exemplary and different numbers for the number of samples in the rolling average could be selected, such as five, ten, twelve, or twenty. Furthermore, the tolerance of 1 degree centigrade is configurable and could be, for example, 2 degrees, 3 degrees etc. Excretion event may be confirmed or detected by accelerometer readings indicating a freefall event.

It is noted that, on occasion, there may be no change in temperature at ingestion or excretion. The processing may include a backup algorithm which is performed in the event the earliest environmental temperature readings at initiation (it being assumed that the capsule 10 has not yet been ingested) are within the threshold range of the expected temperature for the environment at the start of the GI tract of the subject mammal. The backup algorithm looks for other ingestion indicators or excretion indicators in recorded readings from other sensors (such as the accelerometer 19 or the reflectometer, and/or the other ingestion indicators or excretion indicators discussed above) that may indicate an excretion or ingestion event. Alternatively, in instances in which the environmental sensor 14 further comprises an environmental humidity sensor 14b, the relative humidity readings may be used as a fallback for temperature. A further example is a manual button press on a user interface of a device (such as the receiver apparatus 30). Embodiments may combine indicators in a hierarchical manner (i.e. look for indicator in temperature readings first, and look for indicators in readings from other sensors only if indicator in temperature readings cannot be found), or may treat indicators equally (i.e. look for any two contemporaneous indicators). Other algorithms for determining timing may be implemented, for example, a confidence level may be attributed to a detected indicator and then only if the confidence level does not satisfy a threshold are readings from other sensors processed to find a contemporaneous indicator to improve confidence. It is noted in this document that humidity refers to relative humidity.

A specific excretion indicator which may be used to add confidence to an excretion indicator (start of decrease from body temperature) in environmental temperature readings is a button press on a bowel movement button on the user interface of the receiver apparatus or coupled smartphone.

At S104a recorded readings later than the determined ingestion event timing are analysed for a gastric-duodenal transition indicator.

The first transition event is gastric emptying or crossing the interface between the stomach and the duodenum. Gastric duodenal indicator or indicators may be detected in a first subset of recorded readings, the first subset being defined temporally by starting after an ingestion event. Furthermore, the first subset may be constrained by sensor, comprising readings from the TCD gas sensor 131. The first subset may further comprise readings from the reflectometer (i.e. the antenna 17 and directional coupler 171) and/or the accelerometer 19.

The gastric-duodenal transition indicator in the TCD gas sensor readings may be a, spike, step change or an inflection point in the TCD gas sensor readings. A correction may be applied to the TCD gas sensor readings to account for changes in environmental temperature, based on recorded readings from the environmental temperature sensor 14a. The correction may be applied at the detecting stage S104a so that the recorded readings themselves are corrected to account from changes in environmental temperature, and a gastric-duodenal transition indicator is detected in the corrected readings. Alternatively, the gastric-duodenal transition indicator may be detected in the raw readings (i.e. the uncorrected readings) and then at the determining step S104 a check performed to determine whether or not the indicator is attributable to a change in the environmental temperature or not, and if not, then it is either determined that the gastric-duodenal transition indicator is caused by a gastric-duodenal transition by the capsule 10, or a further condition is applied in the determination (for example, recorded readings from another sensor are checked for a contemporaneous indicator). Alternatively, the further condition may be a threshold or some other condition applied to the detected spike, step change, or inflection point itself.

The primary physical mechanism being sensed in the TCD gas sensor readings in detecting the gastric-duodenal transition indicator is as follows: Hydrochloric acid in the gastric juices leaving the stomach mixes with bicarbonate within the bile acids that is released by the pancreas. This bile acid works to neutralize the pH of the liquid and a by-product of this reaction is CO2. In this area of the GI tract the surrounding gases are primarily N2 and O2 with some trace amounts of CO2. The amount of CO2 created in this reaction are significantly higher than the trace amounts that are around due to swallowing of exhaled breath. Therefore, simply using the TCD sensor output without calculating CO2 is appropriate. In other words, the TCD gas sensor readings, once corrected for environmental temperature variations, themselves provide the gastric-duodenal transition indicator, owing to a change in heat conductivity caused by variation in CO2 concentration across the two sides of the gastric-duodenal transition. For motility purposes (i.e. for determining the location of the ingestible capsule 10) there is no particular need to calculate the actual CO2 concentration.

As the TCD sensor 131 is affected by the temperature of the gas mixture at the location of the capsule, a temperature correction process is required to account for changes in the external environmental temperature changes i.e. drinking cold water, exercise, eating etc. Starting from the determined ingestion event timing, the first bump, step change or large inflection in the readings of the TCD gas sensor 131 plotted against time, that is not associated with an environmental temperature change, identifies the gastric-duodenal transition.

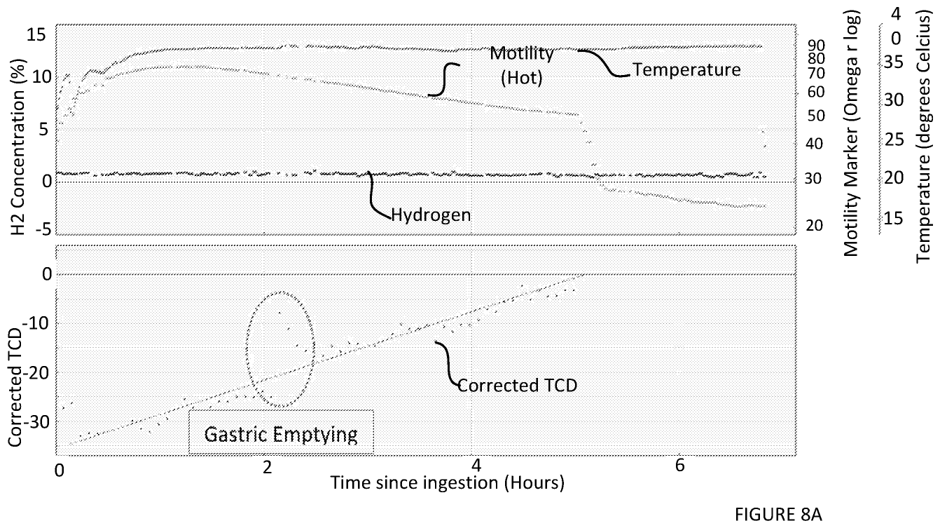

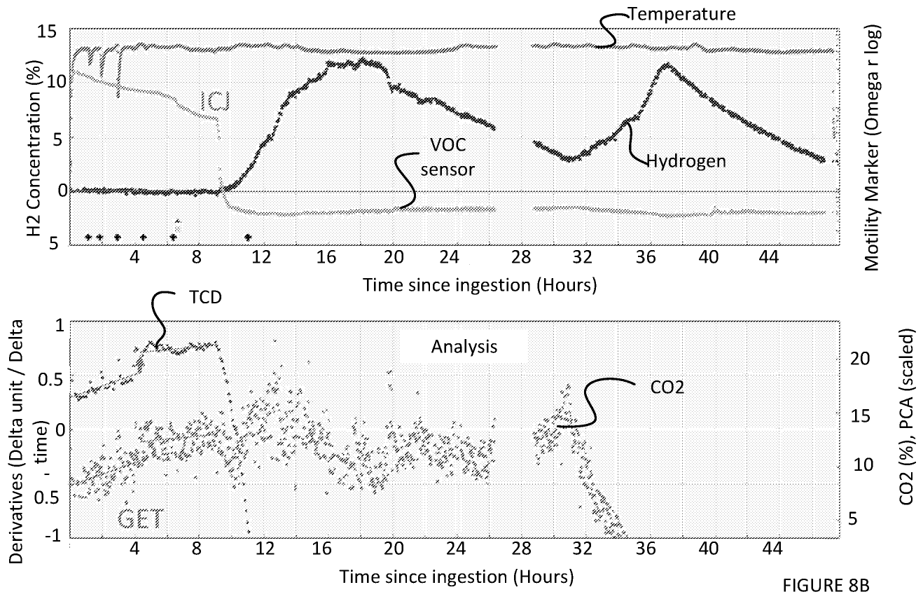

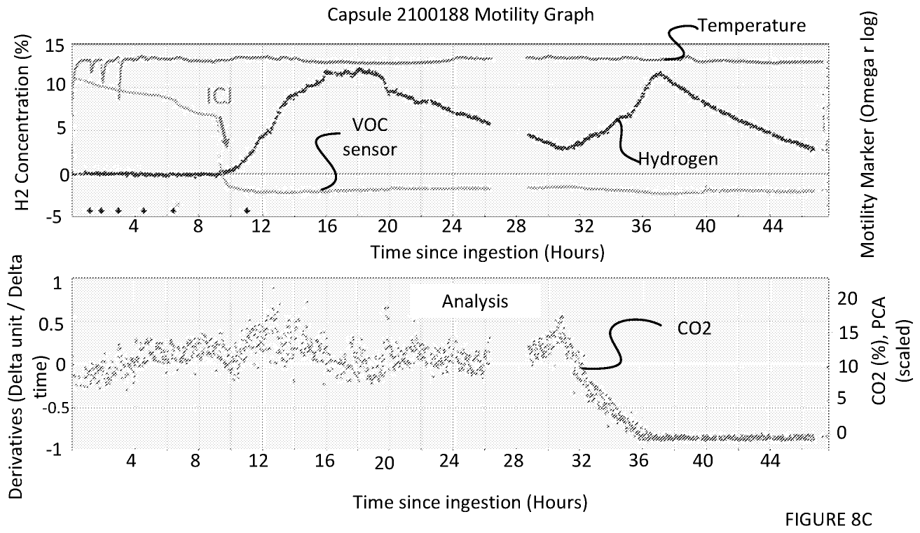

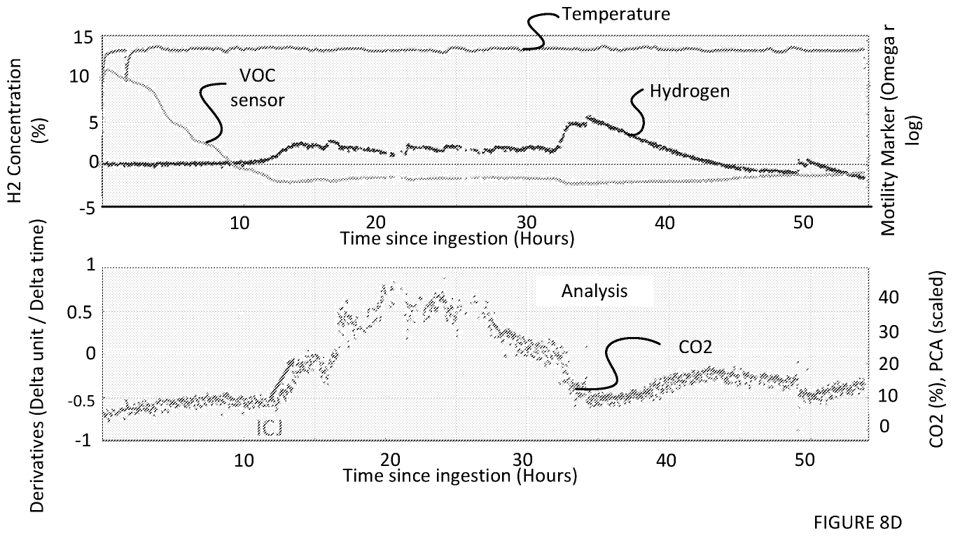

FIG. 8A illustrates recorded readings of an environmental temperature sensor 14a (top line of readings on the top graph) against time, and corrected TCD gas sensor readings against time for an instance of capsule ingestion and progression through a GI tract. The gastric-duodenal transition indicator, which may be labelled gastric emptying, is indicated by a spike above a threshold height in the corrected TCD gas sensor readings. Spike height may be measured, for example, by distance (e.g. as a proportion, as an absolute value, or as a number of standard deviations) from a trend line fitted against the readings up to that point, or from an average value up to that point (wherein the processor maintains an average value).

FIG. 8B shows gastric emptying as visible in TCD sensor output and CO2 readings. CO2 is produced when the hydrochloric acid in the gastric juices leave the stomach and mix with bicarbonate in the bile acids released by the pancreas. This reaction also neutralizes the pH of the liquid. Embodiments use the temperature compensated raw TCD sensor output to detect this event, rather than the calculated CO2, since it contains much less noise. The TCD sensor output is adjusted to compensate for the temperature fluctuations measured by the environmental temperature sensor 14a. An algorithm is used to find the moment CO2 increases by removing drinking events and searching for a distinct discontinuity in the TCD output between ingestion and ICJ transition.

The on-board S104a processing may include detecting, as a first gastric-duodenal transition indicator, a gastric-duodenal transition indicator in the TCD gas sensor readings from the first subset of recorded readings. The determining S104 (which may be performed off-board or on-board) may include calculating a confidence score representing a likelihood that the detected gastric-duodenal transition indicator in the TCD gas sensor readings is caused by the ingestible capsule 10 traversing the gastric-duodenal junction. The confidence score may be based, for example, on the height of the spike relative to the trend line, wherein more standard deviations above the trend line gives higher confidence level. A probability distribution lookup table may be utilised to transform spike height to confidence score. The confidence score may be a percentage likelihood of the spike in corrected TCD readings being caused by a first transition event rather than being caused by noise or other random variation in the corrected TCD readings.

The determination processing S104 may include comparing the calculated confidence score with a threshold, and if the confidence score meets the threshold, determining that the first transition event has occurred and a timing thereof based on a timing of the detected gastric-duodenal transition indicator, and if the confidence score does not meet the threshold, assigning the detected gastric-duodenal transition indicator from the TCD gas sensor readings as a first gastric-duodenal transition indicator, and detecting whether or not a second gastric-duodenal transition indicator is present in readings from the first subset other than the TCD gas sensor readings and contemporaneous with the first gastric-duodenal transition indicator, and if the second gastric-duodenal transition indicator is detected, determining that the first transition event has occurred and a timing thereof based on a timing of the first gastric-duodenal transition indicator.

In effect, the first gastric-duodenal transition indicator not meeting the confidence score threshold may initiate a further processing thread for detecting a further gastric-duodenal transition indicator to add confidence to the first. Recorded readings contemporaneous with the first gastric duodenal transition indicator from other sensors or pseudo sensors are analysed to identify one or more second gastric-duodenal transition indicators. The temporal bounds of the readings included in the analysis may be, for example, a predefined temporal distance either side of the first gastric duodenal transition indicator, for example, one second, five seconds, ten seconds, twenty seconds, thirty seconds, one minute, two minute, or five minutes. Recorded readings from either or both of the reflectometer (i.e. the antenna 17 and directional coupler 171 configured as a reflectometer sensing whether and how the dielectric of the environment surrounding the capsule 10 changes) and the accelerometer 19 (i.e. sensing whether and how the capsule rate of orientation change varies) may be processed in seeking to identify the one or more second gastric-duodenal transition indicators.

As illustrated in FIG. 2, the circuitry includes a directional coupler 171 in series with the antenna 17, which operate as a reflectometer. A diode detector measures the amplitude of reflected signals from the antenna. The measurements of the diode detector are the reflectometer readings, and measure the reflected energy from the antenna, i.e. energy that was not radiated from the antenna 17 due to impedance mismatches. The reflectometer readings measure the antenna's radiation efficiency which is affected by the dielectric of the material surrounding the capsule.

The readings may become noisy and/or a baseline shift occurs at the timing of the gastric-duodenal transition event. For example, the increase in noise and/or the baseline shift are detectable as transition indicators.

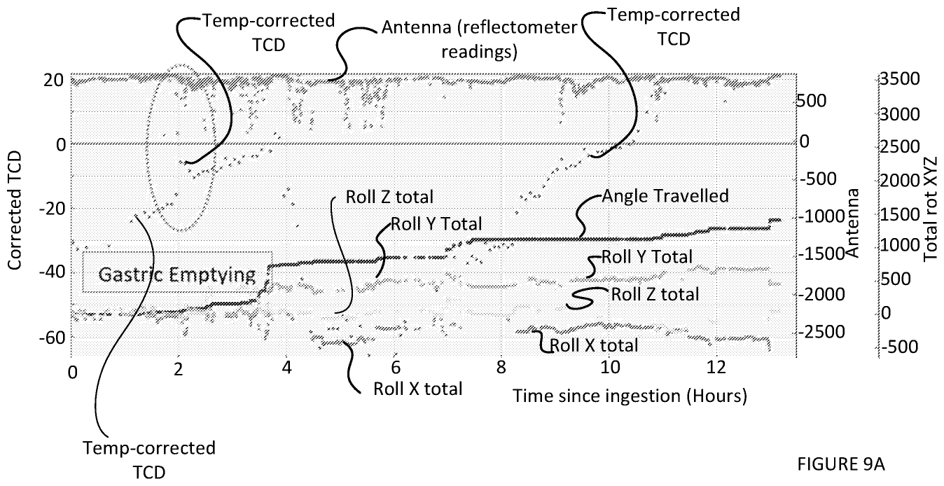

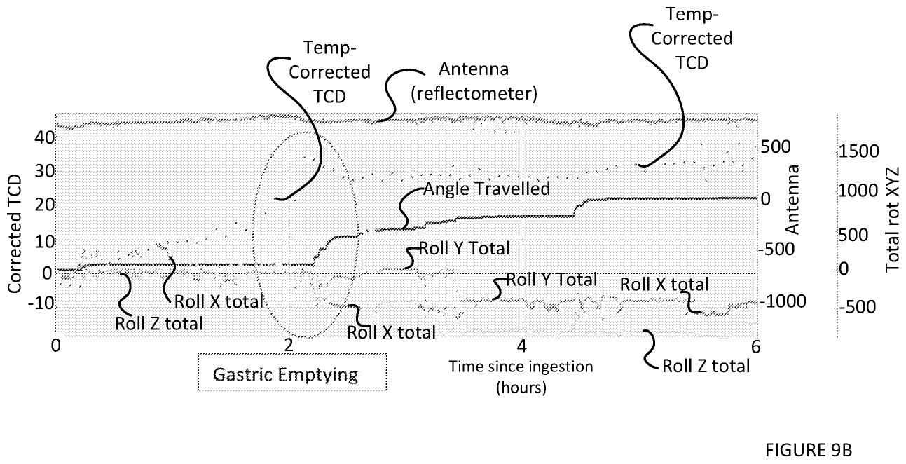

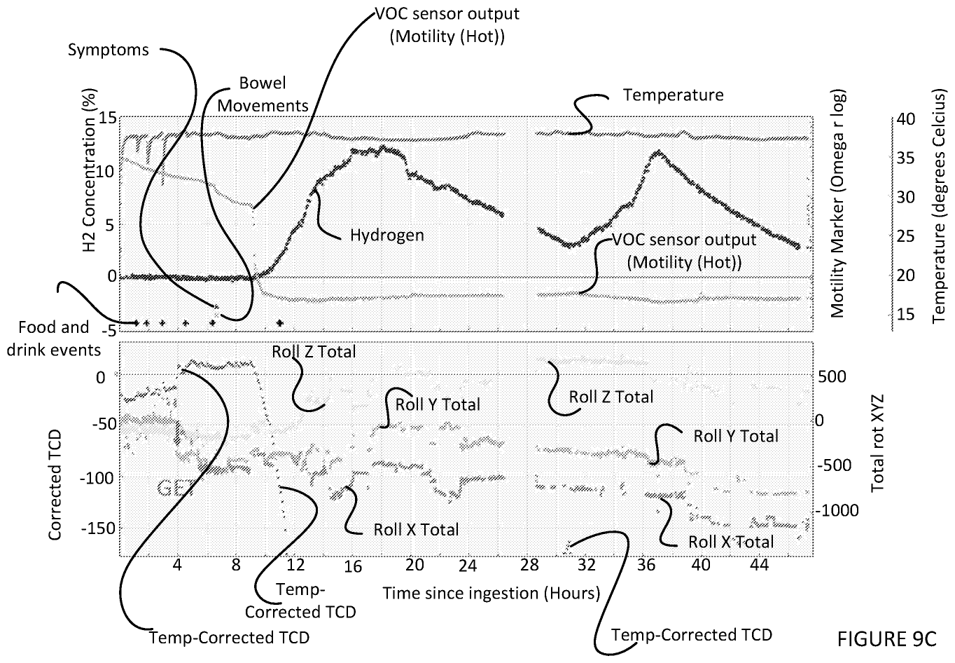

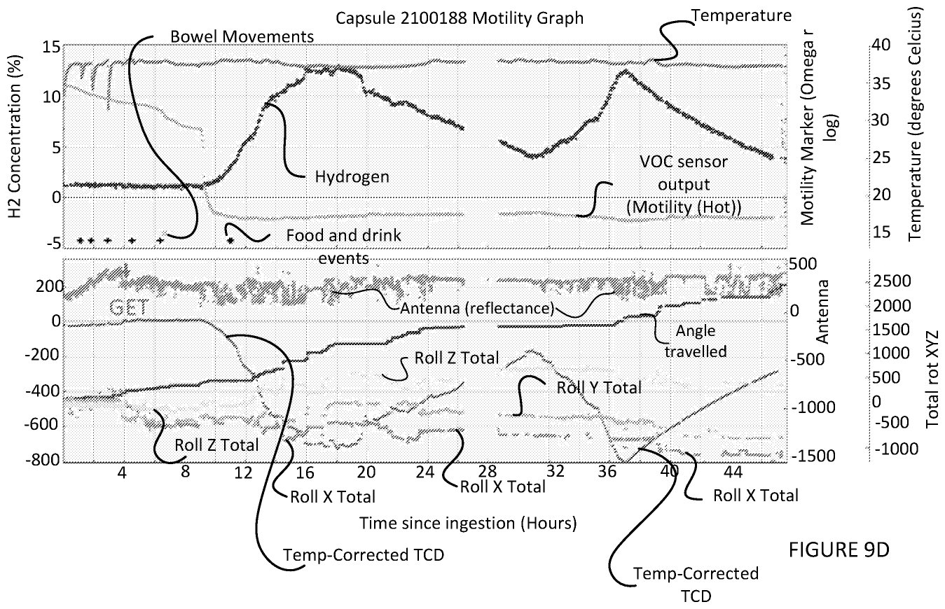

FIG. 9D illustrates (on the uppermost plot on the lower of the two sets of axes) reflectometer readings against time (labelled “Ant” for antenna), and is marked with the gastric emptying event. The antenna 17 and directional coupler 171 function as a reflectometer to measure the reflected energy from the antenna, i.e. energy that wasn't radiated out of the antenna. This signal varies as the surrounding dielectric properties change, most notably when the capsule leaves the cavernous fluid filled stomach and transitions to being surrounded by tubular tissue in the small intestine. A shift in the reflectometer readings is observed to be coincident with the TCD marker, adding confidence, as a secondary measure.

FIG. 9A is a plot of recorded readings (or processed versions thereof) against time for a number of sensors and pseudo sensors in the capsule 10. A gastric emptying (gastric-duodenal transition) event is labelled. The top plot in the graph of FIG. 9A is reflectometer readings against time (labelled “Ant” for antenna). It can be seen that a baseline shift occurs at a time coincident with the spike in corrected TCD gas sensor readings. So, if, for example, a confidence score representing likelihood of the spike being caused by gastric-duodenal transition did not meet a threshold, then the readings of the reflectometer are analysed to detect a baseline shift coincident with the spike. For example, a baseline shift may be detected by, on a progressive/rolling basis, comparing a mean value of a latest number (e.g. five, ten, or twenty) of consecutive readings, with a mean value of a number of readings preceding (or proceeding in the case of reverse chronological processing) the latest number of consecutive readings. A baseline shift may be indicated by a difference more than a threshold, wherein the threshold may be an absolute value, a proportion, or determined relative to a standard deviation in the readings. Detecting a coincidental gastric-duodenal indicator in the output of the reflectometer may be sufficient to confirm that the first gastric duodenal transition indicator is caused by gastric-duodenal transition of the capsule 10 and thus to determine the timing of the gastric-duodenal transition. Alternatively, the combination of the two indicators may be assessed via a probability model to revise the confidence score and compare the revised confidence score with a threshold, wherein meeting the threshold is to determine that the first gastric duodenal transition indicator is caused by gastric-duodenal transition of the capsule 10 and thus to determine the timing of the gastric-duodenal transition.

An exemplary accelerometer 19 measures roll about three mutually orthogonal axes. The readings from the accelerometer 19 may be vectors with a component per axis, with each component indicating an instantaneous angular acceleration about the corresponding axis, or an average acceleration about the corresponding axis over the time period since the preceding reading. Alternatively, the readings may give a three dimensional orientation of the capsule. On-board the capsule, at a receiver apparatus 30 or at a remote computer 20, processing of the readings from the accelerometer may be performed to generate a representation (such as a plot vs time) of aggregated (i.e. all three axes) accelerometer readings from which a marker (i.e. a gastro-duodenal transition indicator) is identifiable. Such a plot or representation may also be used to identify markers for other events including excretion event. In FIG. 9A, an “angle travelled” plot is generated. It is an accumulation of scalar angular displacement about all three axes cumulatively over time, wherein a low pass filter is applied to filter out small angular displacements. Angle travelled is an exemplary metric that may be calculated periodically to represent the accelerometer data, with the periodical calculated values being included in the data transmission payload in place of the relatively larger data load of the raw accelerometer data.

FIG. 9C shows roll in each of three mutually orthogonal dimensions and is marked with gastric emptying event, from which it can be seen that the change in accelerometer readings correlates temporally with the change in corrected TCD readings (i.e. can be used to add confidence to a detection of gastric-duodenal transition indicator in the temperature corrected TCD readings). The capsule orientation is measured using a triaxial accelerometer and tracking the gravity vector with respect the capsule frame of reference. When the capsule leaves the stomach it tends to experience rapid changes in its orientation as it transits through the duodenum and small intestine. “Angle Travelled”, simply accumulates the orientation change in excess of a 90 degree hysteresis angle. This processing technique tends to be robust to small changes in orientation experienced in the stomach and avoids some of the complexities of other approaches.

A first technique for processing accelerometer data may be referred to as angle travelled. Angle travelled uses vector mathematics to calculate the angle between the gravity vector and a temporary vector. The temporary vector is pulled in the direction of the change in angle, only when this angle exceeds a given threshold (currently 90 Deg). It is then the accumulation of the change in the temporary vector that is visualized in the representation from which markers are identifiable. What is generally seen is that this measure does not change much in the stomach since the angle between the gravity and temporary vectors rarely exceed the threshold in any one direction, (small back and forth orientation changes in the stomach are effectively ignored by the inherent hysteresis of this algorithm) and that once in the tortuous lumen of the small intestine, this measure accumulates significantly due to the larger, more continuous orientation changes of the capsule. Thus, a step change in the cumulative angle travelled measure is a gastric-duodenal transition indicator, and may be detected on-board the capsule or off-board.

In an exemplary implementation of angle travelled: the accelerometer readings may provide a reading of an orientation of the ingestible capsule relative to a frame of reference in fixed relation to a gravitational vector. Processing of the readings from the accelerometer may comprise recording an orientation of the ingestible capsule given by a first accelerometer reading as a reference orientation, and repetitively in respect of each successive accelerometer reading chronologically: determining whether the orientation of the ingestible capsule given by the respective accelerometer reading is more than a threshold angular displacement from the reference orientation, and if the threshold angular displacement is not met, progressing to the next accelerometer reading without changing the reference orientation, and if the threshold angular displacement is met, changing the reference orientation to align with the orientation of the ingestible capsule given by the respective accelerometer reading. An indicator, such as the gastric-duodenal transition indicator, may be a step change in the rate of change of the reference orientation.

FIG. 9B indicates that a step change in a plot of angle travelled is identifiable within a threshold time period of the detected spike in the TCD gas sensor readings. Therefore, the step change in the plot of angle travelled increases confidence in the hypothesis that the detected spike in the TCD gas sensor readings is caused by gastric-duodenal transition. There are two approximately contemporaneous gastric-duodenal transition indicators, which enables the timing of one of the indicators (which one may be pre-selected, for example, the TCD gas sensor readings) to be determined as the timing of the transition event.

A second technique for processing accelerometer data may be referred to as total roll. Total roll calculates the angle between the gravity vector and each of the capsule X, Y and Z axes and expresses this as a continuous measure that can accumulate beyond 360 Deg. For example, if the capsule x axis is at an angle of 350 Deg and rotates by a further 20 Deg, the resulting angle is expressed as 370 Deg rather than 10 Deg. This helps when representing the readings as a plot from which markers are identified since it avoids the sudden angle changes associated with crossing the zero line. In the example a real change of 20 Deg would be visualized instead of an artificial change of 340 Deg. In addition to this basic approach, low pass filtering may be applied to filter the raw data to remove sensor noise. Additionally, angles are only calculated when the raw accelerometer data provide sufficient data to calculate a meaningful angle. An example of where this is not the case is when the two accelerometer axis values used to calculate the orientation angle around the third axis both approach zero. In this case the calculation will be dominated by sensor noise and so a meaningful angle cannot be determined.

The accelerometer readings provide a reading of an orientation of the ingestible capsule relative to a frame of reference in fixed relation to a gravitational vector. Exemplary processing of the readings from the accelerometer may comprise for each of three orthogonal axes in fixed spatial relation to the ingestible capsule derivable from the reading of the orientation, repetitively in respect of each successive accelerometer reading chronologically: calculating, as a scalar value, a change in the orthogonal axis relative to the gravitational vector from the preceding accelerometer reading; applying a low pass filter to the calculated changes; recording the cumulative filtered calculated changes. A marker serving as a gastric-duodenal transition indicator may be, for example, an increase (such as a spike or step change) in the rate of increase in the cumulative filtered calculated changes.

In the case of chronological processing, at S106a signals later than the determined first transition event timing are analysed for an ileocecal junction transition indicator, or possibly signals later than the determined ingestion event timing (for example if S104 is to be performed off-board and S106a is to be performed on-board).

The second transition event is passage of the capsule 10 through the ileocecal junction. Ileocecal junction transition indicator (or ileocecal junction indicator) or indicators may be detected in a second subset of recorded readings, the second subset being defined temporally by a preceding determined event timing. Furthermore, the second subset may be constrained by sensor, comprising readings from the sensor side of the VOC gas sensor 132a.

The ileocecal junction transition indicator in the VOC gas sensor readings may be a spike, step change or an inflection point in the VOC gas sensor readings. Spike may be detectable via comparison of a most recent signal reading with an average-to-date value, wherein a predefined number of adjacent readings exceed one another and exceed the average-to-date by more than a predefined threshold is defined as a spike, for example. An inflection point is detectable by monitoring gradients and identifying when a second derivative (i.e. rate of change of gradient) changes from positive to negative or vice-versa. A step change may be detectable via comparison of a most recent signal reading with an average-to-date value, wherein a predefined number of adjacent readings exceed the average-to-date by more than a predefined threshold is defined as a spike, for example.

The determining second transition event timing S106 is an application of one or more conditions to the detected ileocecal junction transition indicator to determine whether or not it can be attributed to (i.e. to predict to within a predefined confidence level) passage of the capsule 10 across the ileocecal junction. The detecting step S106a may be performed on-board and the determining step S106 performed off-board, or both may be performed on-board. In case the detecting step S106a is performed on-board and the determining S106 off-board, the detected indicator or a characterisation thereof is added to the data transmission payload, optionally along with contemporaneous readings from other sensors.

The transit prediction of the transition from small intestine to large intestine is the determined second transition event timing. The gas environment change between the small and large intestine is significant due to the large intestine's bacterial population occurring in significantly higher prevalence, driving the creation, or increase, in volatiles and a reduction on O2 through fermentation of carbohydrates and proteins by the microbiota.

The VOC gas sensor output 132 from the sensor side 132a is sensitive to many different volatile analytes with the largest response being due to H2, and O2. At the time of transition through the ileocecal valve a large reduction on the VOC sensor is observed. As the capsule transits the GI tract the environment is increasingly anaerobic as the O2 is consumed by bacteria. FIG. 8C illustrates indicators of ICJ on plots of VOC sensor output and determined H2 concentration. The indicator in the VOC sensor output may be identified at S106a through plotting the differential of the VOC sensor side readings vs time whilst the sensor is heated and finding the tallest negative peak. This differential locates the point of greatest change which is associated with the transition but does not occur at the start of the transition event. The start of the transition event may be found by the initial inflection point from the baseline in the first derivative. Thus, the indicator may detected by the tallest negative peak, and the event timing determined by the inflection point. The tallest negative peak may be found retrospectively by analyzing VOC gas sensor readings from a predefined temporal period (e.g. one hour, two hours, four hours etc) following determined gastric-duodenal transition event timing, or preceding the determined excretion event timing (in the case of reverse-chronological processing). Alternatively, a threshold negative peak size may be determined, with the first peak exceeding the threshold size being detected as the ileocecal junction transition indicator.

As illustrated in FIG. 8C, an ICJ indicator is also present in the determined H2 concentration percentage, as a sharp increase in H2 when the capsule reaches the colon. The H2 produced in the GI tract is a byproduct of fermentation. The colonies of bacteria are orders of magnitude larger in the colon than in the small bowel. Therefore, determined H2 concentration may be used to add confidence to the ileocecal junction transition indicator in the VOC sensor output. H2 concentration may be sensed directly, such as by a dedicated H2 gas sensor, or may be derived from gas sensors, for example by taking TCD gas sensor readings at different operating temperature setpoints.

FIG. 8D illustrates a further indicator for ileocecal junction transition in the form of the detected CO2 concentration. CO2 in the GI tract is produced as a byproduct of fermentation. The colonies of bacteria are orders of magnitude larger in the colon than in the small bowel. Therefore, determined CO2 concentration may be used as an ileocecal junction transition indicator in itself, or to add confidence to another ileocecal junction transition indicator.

In general the temperature drop at excretion is a reliable signal. However, there are cases when the temperature drop is not observed in the data. Determining the excretion event timing S107 may include comparing the relative humidity of one or more readings with an expected relative humidity for the environment at the end of the GI tract of the subject mammal 40, wherein a change of more than a threshold higher (in the case of reverse chronological processing) or lower (in the case of chronological processing) is a determination that the capsule 10 has been excreted. Alternatively the condition may be that a predefined number or more consecutive readings are outside of a threshold of the expected relative humidity for the environment at the end of the GI tract of the subject mammal.

It is noted that, on occasion, there may be no change in temperature at excretion. The processing may include a backup algorithm which is performed in the event the earliest environmental temperature readings at initiation (it being assumed that the capsule 10 has not yet been ingested) are within the threshold range of the expected temperature for the environment at the end of the GI tract of the subject mammal (which would be an indication that the subject is in an environment with a temperature at or around the expected GI tract temperature). The backup algorithm looks for markers in recorded readings from other sensors that may indicate an excretion event. Since excretion is generally associated with a physical fall, the marker may be an indicator in the accelerometer readings. Alternatively or additionally, a change in relative humidity may be detected by the backup algorithm.

The determination that the excretion event has occurred at S107 triggers a Bluetooth beacon transmission mode which transmits some or all of the data transmission payload that has not yet been transmitted. A receiver apparatus 30, which may be a dedicated receiver apparatus or may be a general purpose device such as a Bluetooth enabled smartphone or tablet device.

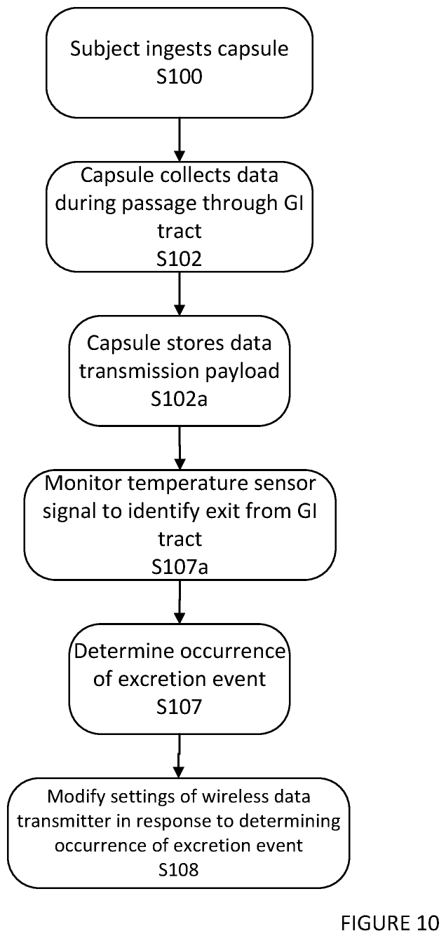

FIG. 10 illustrates a method or process performed by an ingestible capsule 10. For example, the ingestible capsule 10 may comprise an ingestible indigestible bio-compatible housing 11; and further comprise, within the housing: a power source 16; sensor hardware including a temperature sensor 14a configured to output a temperature sensor signal representing a temperature of an environment surrounding the ingestible capsule 10; processor hardware 151; memory hardware 152; and a wireless data transmitter 18. The processor hardware 151 may be a CPU, processor, or a microprocessor. The memory hardware 152 is a chip configured to store data. The memory hardware 152 and the processor hardware 151 may be provided as part of a single microcontroller integrated chip.

The memory hardware 152 may store software, a computer program, or processing instructions, which, when executed by the processor hardware 151, cause the processor hardware to execute the method or process as illustrated in FIG. 10. The software, computer program, or processing instructions stored by the memory hardware may also cause the processor hardware 151 to perform the other functions attributed to the processor hardware 151 elsewhere in the present disclosure. The memory hardware 152 is exemplary of a computer-readable medium.

At S100 the capsule 10 is ingested by a subject mammal 40. The subject mammal 40 may be a human. Following ingestion, at S102 the capsule 10 is configured to collect data during a passage through a GI tract of the subject mammal 40. The data is collected at the capsule 10 and specifically at the memory hardware 152 by processing at the processor hardware 151 signals received from one or more sensors forming the sensor hardware of the capsule 10.

At S102a the processor hardware 151 is configured to receive a signal output by the sensor hardware, to process the received signal, and to store some or all of the processed signal, or data extracted therefrom, on the memory hardware as data transmission payload. The data transmission payload may comprise a metric representing a signal from a single sensor or from plural sensors. The data transmission payload may comprise one or more motility indicators or diagnostic indicators, as discussed elsewhere in the present disclosure. The data transmission payload may comprise a report of one or a series of events determined to have occurred based on one or more identified motility markers. The data transmission payload may also include information such as remaining capacity of power source 16.

At S107a the processor hardware 151 is configured to receive and monitor the temperature sensor signal to identify when the temperature sensor signal indicates that the ingestible capsule 10 ceases to be within the GI tract of the subject mammal 40. For example, the processor hardware 151 may compare a temperature represented by the temperature sensor signal and to determine when the said temperature ceases to be within a predefined range for the GI tract of the subject mammal 40. In particular, the monitoring may begin following determination by the ingestible capsule 10 that the ingestible capsule 10 is beyond the stomach of the subject mammal 40, i.e. that a gastric-duodenal transition event has occurred.

At S107, the processor hardware 151 is configured to determine that the excretion event has occurred. For example, the identifying step S107a may identify a drop in the temperature represented by the temperature sensor signal, and at S107 the processor hardware 151 checks whether or not gastric-duodenal transition has been determined to have occurred already, and if not to determine that excretion has not occurred and to continue the monitoring and identifying step S107a, and if so, to determine that the identified drop in temperature does represent an excretion event and that excretion has occurred. It is noted that the capsule 10 may be configured in such a way that for a set of readings to be detected as an indicator of capsule excretion is necessarily also a determination that excretion has occurred and thus that the timing of the indicator is the timing of excretion. In other words, the determination step S107 may be integrated into the processing of the detecting step S107a.

At S108, the processor hardware 151, being a microcontroller or otherwise, is configured, in response to determining occurrence of the excretion event, to modify one or more settings of the wireless data transmitter to start, restart, or increase a rate of, wireless transmission of the data transmission payload away from the capsule by the wireless data transmitter. For example, the wireless data transmitter 18 may be a Bluetooth transceiver. For example, modifying the settings may be to increase a rate of data transmission. Modifying the settings may be to re-connect with a receiver device 30 exterior to the subject mammal 40. The receiver device 30 may be a Bluetooth-enabled communications device such as a smartphone or a tablet computer. Modifying the settings may cause the wireless data transmitter 18 to transmit in a broadcast or inquiry mode a report that occurrence of the excretion event has been determined to a receiver device 30, and to wirelessly connect, or to wirelessly re-connect following an initial connection pre-ingestion of the ingestible capsule, to the receiver device 30 to transmit the remainder of the data transmission payload.

Modifying transmission settings after detection of excretion may comprise increasing transmission power. The rationale is as follows:
Post excretion is the last chance to get the data away from the capsule 10 before it is flushed away.
The complete dataset is available which enables the calculation of all the transit metrics and any other items like peak H2 time and total H2 (area under the curve) etc. (resulting in a potentially smaller data transmission payload)
The capsule 10 no longer being resident in the body means the radio power can be increased without exceeding Specific Absorption Rate (SAR) safety limits
The bowl environment is likely to be more consistent than the variation in patient BMI's and compliance.

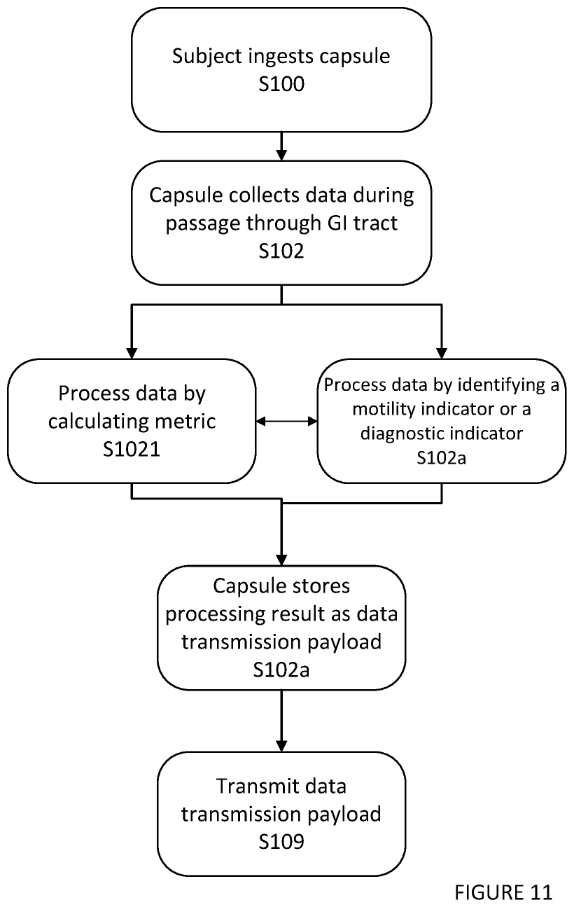

FIG. 11 illustrates a method or process performed by an ingestible capsule 10. For example, the ingestible capsule may comprise an ingestible indigestible bio-compatible housing 11; and further comprise, within the housing: a power source 16; sensor hardware; processor hardware 151; memory hardware 152; and a Bluetooth transceiver 18 or a wireless data transmitter 18 configured to transmit according to a communications protocol other than Bluetooth, such as Wi-fi. The processor hardware 151 may be a CPU, processor, or a microprocessor. The memory hardware 152 is a chip configured to store data. The memory hardware 152 and the processor hardware 151 may be provided as part of a single microcontroller integrated chip.

The memory hardware 152 may store software, a computer program, or processing instructions, which, when executed by the processor hardware 151, cause the processor hardware to execute the method or process as illustrated in FIG. 11. The software, computer program, or processing instructions stored by the memory hardware may also cause the processor hardware 151 to perform the other functions attributed to the processor hardware 151 elsewhere in the present disclosure. The memory hardware 152 is exemplary of a computer-readable medium.

At S100 the capsule 10 is ingested by a subject mammal 40. The subject mammal 40 may be a human. Following ingestion, at S102 the capsule 10 is configured to collect data during a passage through a GI tract of the subject mammal 40. The data is collected at the capsule 10 and specifically at the memory hardware 152 by processing at the processor hardware 151 signals received from one or more sensors forming the sensor hardware of the capsule 10.

The processor hardware 151 executes one or both of steps S1021 and S102a. In the case in which both are performed, it may be that data generated by step S1021 is processed in step S102a, or that data generated by step S102a is processed in step S1021, as indicated by the double-ended arrow in FIG. 11.

Steps S1021 and S102a represent different data processing functions that may be performed by the processor hardware 151. The processor hardware 151 is configured to receive a signal output by the sensor hardware (i.e. signals or readings from one or more sensors), to process the received signal by S1021 calculating a metric representing the received signal or signals, or S102a by identifying a motility indicator or a diagnostic indicator in the received signal. A metric may be a maximum, minimum, or a local maximum or minimum bounded by one or more determined motility events. Metrics may be calculated in accordance with calibration tables or calibration parameters. A motility indicator or a diagnostic indicator is a predefined pattern, value range, spike, step change, inflection point, local maximum or local minimum, predefined change or sequence of changes, in a signal or signals output by one or more sensors among the sensor hardware. A motility indicator indicates that a motility event has occurred. Motility events include one or more from among: ingestion, excretion, gastric-duodenal transition, ileocecal junction transition. A diagnostic indicator indicates that the subject mammal has a specific medical ailment such as a condition or a disease. Examples include gastroparesis and small intestinal bacterial overgrowth. Indicators, whether of motility or diagnostics, refer to signatures or other characteristic features of signals or readings from sensors. Further processing such as comparison with a threshold or some other form of confidence testing may be required (and performed by the processor hardware 151 or in subsequent off-board processing) to determine whether the indicator is caused by the motility event or medical condition, as appropriate.

At step S102a the capsule 10 stores the processing results either on the memory hardware 152 or optionally on a buffer of the Bluetooth transceiver, as all or part of a data transmission payload. At S109 the data transmission payload is transmitted away from the Bluetooth transceiver 18 to a receiver device 30 external to the subject mammal 40 and external to the capsule 10. At least a portion of the data transmission payload is transmitted while the capsule 10 is still in the GI tract of the subject mammal 40. Optionally, a further portion may be transmitted post-excretion such as illustrated at steps S107 and S108 of FIG. 10 and discussed in further detail above.

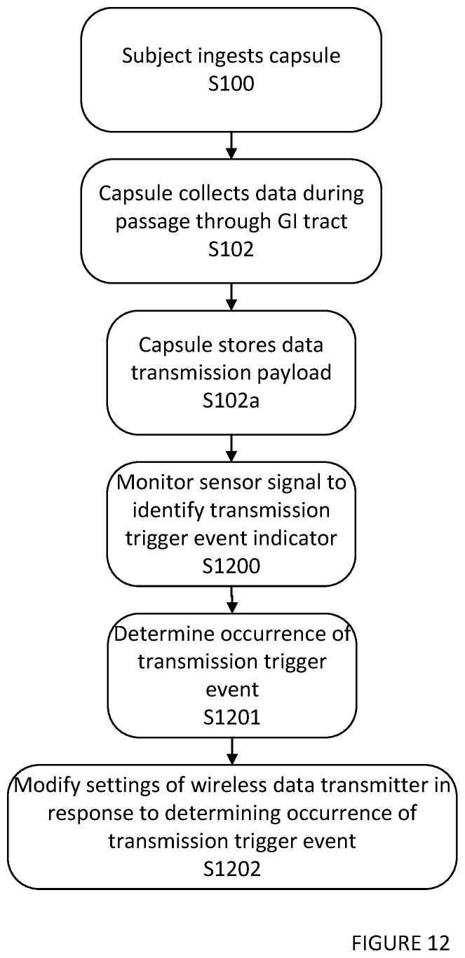

FIG. 12 illustrates a method or process performed by an ingestible capsule 10. For example, the ingestible capsule may comprise an ingestible indigestible bio-compatible housing 11; and further comprise, within the housing: a power source 16; sensor hardware; processor hardware 151; memory hardware 152; and a Bluetooth transceiver 18 or a wireless data transmitter 18 configured to transmit according to a communications protocol other than Bluetooth, such as Wi-fi. The processor hardware 151 may be a CPU, processor, or a microprocessor. The memory hardware 152 is a chip configured to store data. The memory hardware 152 and the processor hardware 151 may be provided as part of a single microcontroller integrated chip.

The memory hardware 152 may store software, a computer program, or processing instructions, which, when executed by the processor hardware 151, cause the processor hardware to execute the method or process as illustrated in FIG. 11. The software, computer program, or processing instructions stored by the memory hardware may also cause the processor hardware 151 to perform the other functions attributed to the processor hardware 151 elsewhere in the present disclosure. The memory hardware 152 is exemplary of a computer-readable medium.

At S100 the capsule 10 is ingested by a subject mammal 40. The subject mammal 40 may be a human. Following ingestion, at S102 the capsule 10 is configured to collect data during a passage through a GI tract of the subject mammal 40. The data is collected at the capsule 10 and specifically at the memory hardware 152 by processing at the processor hardware 151 signals received from one or more sensors forming the sensor hardware of the capsule 10.

The processor hardware 151 executes steps S102 and S102a, as discussed above.

Steps S1200, S1201, and S1202 represent different data processing functions that may be performed by the processor hardware 151. The processor hardware 151 is configured to receive a signal output by the sensor hardware (i.e. signals or readings from one or more sensors), to process the received signal by S1200, for example by calculating a metric representing the received signal or signals, and/or by identifying a motility indicator or a diagnostic indicator in the received signal. A metric may be a maximum, minimum, or a local maximum or minimum bounded by one or more determined motility events. Metrics may be calculated in accordance with calibration tables or calibration parameters. A motility indicator or a diagnostic indicator is a predefined pattern, value range, spike, step change, inflection point, local maximum or local minimum, predefined change or sequence of changes, in a signal or signals output by one or more sensors among the sensor hardware. A motility indicator indicates that a motility event has occurred. Motility events include one or more from among: ingestion, excretion, gastric-duodenal transition, ileocecal junction transition. A diagnostic indicator indicates that the subject mammal has a specific medical condition such as a condition or a disease. Examples include gastroparesis and small intestinal bacterial overgrowth. Indicators, whether of motility or diagnostics, refer to signatures or other characteristic features of signals or readings from sensors. Further processing such as comparison with a threshold or some other form of confidence testing may be required (and performed by the processor hardware 151 or in subsequent off-board processing) to determine whether the indicator is caused by the motility event or medical condition, as appropriate at S1201.

At S1202 the settings of the wireless data transmitter are modified in response to determining the occurrence of the transmission trigger event at S1201. For example, the wireless data transmitter 18 may be a Bluetooth transceiver. For example, modifying the settings may be to increase a rate of data transmission. Modifying the settings may be to re-connect with a receiver device 30 exterior to the subject mammal 40. The receiver device 30 may be a Bluetooth-enabled communications device such as a smartphone or a tablet computer. Modifying the settings may cause the wireless data transmitter 18 to transmit in a broadcast or inquiry mode a report that occurrence of the excretion event has been determined to a receiver device 30, and to wirelessly connect, or to wirelessly re-connect following an initial connection pre-ingestion of the ingestible capsule, to the receiver device 30 to transmit the remainder of the data transmission payload.

Modifying transmission settings after detection of excretion may comprise increasing transmission power, increasing signal intensity, or otherwise proactively modifying settings to increase signal power.

1-85. (canceled)

86. An ingestible capsule comprising:
an ingestible indigestible bio-compatible housing;
and further comprising, within the housing:
a power source;
sensor hardware including a temperature sensor configured to output a temperature sensor signal representing a temperature of an environment surrounding the ingestible capsule;
processor hardware;
memory hardware; and
a wireless data transmitter;
wherein, during a passage of the ingestible capsule through a GI tract of the subject mammal, the processor hardware is configured to receive a signal being output by the sensor hardware, to process the received signal, and to store some or all of the processed signal, or data extracted therefrom, on the memory hardware as data transmission payload;
the processor hardware is configured to receive and monitor the temperature sensor signal to identify when the temperature sensor signal indicates that the ingestible capsule ceases to be within the GI tract of the subject mammal, and in response to said identification to determine occurrence of an excretion event, and in response to determining occurrence of the excretion event to modify one or more settings of the wireless data transmitter to start, restart, increase transmission power of, or increase a rate of, wireless transmission of the data transmission payload away from the ingestible capsule by the wireless data transmitter.

87. The ingestible capsule according to claim 86, wherein the data transmission payload transmitted away from the capsule in response to determining occurrence of the excretion event, comprises a report that occurrence of the excretion event has been determined.

88. The ingestible capsule according to claim 87, wherein modifying the settings causes the wireless data transceiver to transmit in a broadcast or inquiry mode the report that occurrence of the excretion event has been determined to a receiver device, and to wirelessly connect, or to wirelessly re-connect following an initial connection pre-ingestion of the ingestible capsule, to the receiver device to transmit the remainder of the data transmission payload.

89. The ingestible capsule according to claim 86, wherein the sensor hardware includes one or more gas sensors, and therein the signal output by the sensor hardware and processed by the processor hardware includes a gas sensor signal output by the one or more gas sensors, and the data transmission payload includes the processed gas sensor signal or data extracted therefrom.

90. The ingestible capsule according to claim 89, wherein the one or more gas sensors includes one or more from among:
one or more spectrophotometers;
one or more Surface Acoustic Wave sensors;
one or more Bulk Acoustic Resonator Arrays;
one or more VOC gas sensors; and
one or more TCD gas sensors;
each of the one or more gas sensors configured to generate a component gas sensor signal forming part of the gas sensor signal, and wherein processing the received gas sensor signal comprises identifying one or more motility event indicators in the received gas sensor signal, and storing a representation of the identified motility indicators on the memory hardware as data transmission payload.

91. The ingestible capsule according to claim 90, wherein
identifying the one or more motility event indicators comprises monitoring the gas sensor signal received from each of the one or more gas sensors in a most recent time period of predefined duration on a rolling basis to identify a spike, step change, or inflection in the gas sensor signal as the motility indicator.

92. The ingestible capsule according to claim 91, wherein the one or more gas sensors comprises one or more from among a VOC gas sensor and a TCD gas sensor, and the gas sensor signal comprises one or more from among a VOC gas sensor signal and a TCD gas sensor signal, accordingly.

93. The ingestible capsule according to claim 90, wherein the sensor hardware further comprises one or more from among:
an accelerometer;
a reflectometer formed by an antenna in series with a directional coupler, wherein the antenna is an antenna of the data transmitter, the antenna being controlled by the processor to transmit an intermittent or continuous signal from which a reflectometer signal is obtainable;
wherein further to identifying the motility event indicator in the gas sensor signal, the processor hardware is configured to:
store, in association with the motility event indicator in the data transmission payload, a representation of a signal received contemporaneously with the motility event indicator from one or more sensors within the housing from among:
a gas sensor other than the gas sensor providing the signal in which the motility event indicator is detected;
the accelerometer; or
the reflectometer.

94. The ingestible capsule according to claim 92, wherein the representation of the signal is one or more from among:
a recording of the signal,
a recording of the signal downsampled by retaining only one in every more than one readings;
a dimensionally reduced version of the signal;
a recording of a confirmatory marker identified by processing the signal;
a characteristic value of the signal obtained by processing the signal, the characteristic value being an average value, a rate of change, a maximum, a local maximum, a minimum, or a local minimum.

95. The ingestible capsule according to claim 86, wherein the ingestible capsule further comprises an accelerometer, and during passage through the GI tract the processor hardware is configured to receive an accelerometer signal output by the accelerometer, to process the received accelerometer signal, and to store the processed accelerometer signal or a representation thereof on the memory hardware as data transmission payload.

96. The ingestible capsule according to claim 95, wherein to determine occurrence of the excretion event in response to identifying the temperature represented by the temperature sensor signal dropping below the predefined range of temperatures for the subject mammal, the processor hardware is configured to determine whether or not the received accelerometer signal indicates the ingestible capsule experiencing a freefall event, and if it is determined that the received accelerometer signal indicates the ingestible capsule experiencing a freefall event, the processor hardware is configured to determine that the excretion event has occurred.

97. The ingestible capsule according to claim 94, wherein the processor hardware is configured to process the temperature sensor signal, and to store the processed temperature signal or data extracted therefrom on the memory hardware as data transmission payload.

98. The ingestible capsule according to claim 86,
wherein the wireless data transmitter is a Bluetooth transceiver.

99. The ingestible capsule according to claim 98, wherein the wireless data transmitter is a Bluetooth transceiver configured to operate according to a Bluetooth Low Energy Coded PHY transmission protocol.

100. The ingestible capsule according to claim 98,
wherein the Bluetooth transceiver comprises an integrated radio and a microcontroller.

101. The ingestible capsule according to claim 98, wherein
modifying one or more settings of the wireless data transmitter in response to determining occurrence of the excretion event includes controlling the Bluetooth transceiver to transmit the data transmission payload stored on the memory hardware by broadcasting data pending transmission from the data transmission payload to a recipient device irrespective of whether or not the recipient device is paired to the Bluetooth transceiver.

102. The ingestible capsule according to claim 98, wherein
preceding determining the occurrence of the excretion event, the Bluetooth transceiver is configured to pair with a Bluetooth compatible device external to the subject mammal and to transfer to the paired device data comprising or representing one or more from among:
a signal or signals from the sensor hardware;
one or more motility indicators identified by processing signals from the sensor hardware;
one or more identified diagnostic indicators identified by processing signals from the sensor hardware;
information representing remaining capacity of the power source;
a metric calculated by processing a signal from a single sensor among the sensor hardware or by combining signals from plural sensors among the sensor hardware;
calculated gas concentration levels for one or more constituent gases among a gas mixture present in the GI tract, calculated by reference to predefined calibration parameters stored on the memory hardware;
and proceeding determination of occurrence of the excretion event the processor hardware is configured to modify settings of the Bluetooth transceiver to transfer the data transmission payload to the same Bluetooth compatible device, either by continuing the existing pairing, by re-pairing, or in the absence of pairing.

103. An ingestible capsule comprising:
an ingestible indigestible bio-compatible housing;
and further comprising, within the housing:
a power source;
sensor hardware;
processor hardware;
memory hardware; and
a wireless data transmitter;
the ingestible capsule being configured, following ingestion by a subject mammal, to collect data during a passage through a GI tract of the subject mammal, during which passage the processor hardware is configured to receive a signal output by the sensor hardware, to process the received signal, and to store some or all of the processed signal, or data extracted therefrom, on the memory hardware as data transmission payload;
the processor hardware is configured to receive and monitor the signal output by the sensor hardware to identify a transmission trigger event indicator, and in response to said identification to determine occurrence of a transmission trigger event, and in response to determining occurrence of the transmission trigger event to modify one or more settings of the wireless data transmitter to start, restart, increase transmission power of, or increase a rate of, wireless transmission of the data transmission payload away from the capsule by the wireless data transmitter.

104. The ingestible capsule according to claim 103, wherein
the transmission trigger event indicator is a motility event indicator associated with ingestion of the ingestible capsule, gastric-duodenal transition of the ingestible capsule, ileocecal junction transition of the ingestible capsule, or excretion of the ingestible capsule; or
the transmission trigger event indicator is a diagnostic indicator associated with clinical diagnosis of a medical condition.

105. A method in an ingestible capsule, the ingestible capsule comprising:
an ingestible indigestible bio-compatible housing;
and further comprising, within the housing:
a power source;
sensor hardware;
processor hardware;
memory hardware; and
a wireless data transmitter;
the method comprising, following ingestion of the ingestible capsule by a subject mammal, during a passage through a GI tract of the subject mammal, at the processor hardware, receiving a signal being output by the sensor hardware, processing the received signal, and storing some or all of the processed signal, or data extracted therefrom, on the memory hardware as data transmission payload;
at the processor hardware, receiving and monitoring the signal output by the sensor hardware to identify a transmission trigger event indicator, and in response to said identification, determining occurrence of a transmission trigger event, and in response to determining occurrence of the transmission trigger event, modifying one or more settings of the wireless data transmitter to start, restart, increase transmission power of, or increase a rate of, wireless transmission of the data transmission payload away from the capsule by the wireless data transmitter.
